SEQUENCE LISTING (13 sequences)

SEQ ID NO: 1 - protein, 27 aa, Microbacterium arborescens
MSLEQLEALDASSEAAEMAASLGSQSC

SEQ ID NO: 2 - protein, 31 aa, Nocardia terpenica
MIDVTNIAELHELDSTSASAELVASISSNGC

SEQ ID NO: 3 - protein, 31 aa, Nocardia altamirensis
MIDVTNIADLHDIDATSGAAELVASISSNGC

SEQ ID NO: 4 - protein, 31 aa, Artificial Sequence
  variant of the precursor peptide
MIDVTNIADLHDIDATSGAAELVGSISSNGC

SEQ ID NO: 5 - protein, 31 aa, Artificial Sequence
  Variant of the precursor peptide
MIDVTNIADLHDIDATSGAAELVASLSSQGC

SEQ ID NO: 6 - protein, 27 aa, Artificial Sequence
  variant of the precursor peptide
MSLEQLEALDASSEAAEMAGSISSNGC

SEQ ID NO: 7 - protein, 27 aa, Artificial Sequence
  variant of the precursor peptide
MSLEQLEALDASSEAAEMAASLSSQGC

SEQ ID NO: 8 - protein, 31 aa, Tsukamurella sp. 1534
MIDVTDINSLQAIESHSATSELLASVSSSGC

SEQ ID NO: 9 - protein, 30 aa, Streptomyces aureus
MDLTNVIDLQGTEIVADGVELPASGSSEGC

SEQ ID NO: 10 - protein, 30 aa, Streptomyces natalensis
MDLTNVMELQGTEIVADGVELPASTSSDGC

SEQ ID NO: 11 - protein, 30 aa, Nocardiopsis chromatogenes
MDIADVMDLQGEEVVADGVELPASTASDGC

SEQ ID NO: 12 - protein, 30 aa, Nonomuraea candida
MDLANVMDLQGTEIVADGIELPASASSETC

SEQ ID NO: 13 - protein, 27 aa, Microbacterium arborescens strain ND21
MTLEQLEALDASSEAAEMAASLGSQSC

(19) United States
(12) Patent Application Publication
(10) Pub. No.: US 2019/0345199 A1
(43) Pub. Date: Nov. 14, 2019

(54) LIPOLANTHIPEPTIDES AND THEIR USES AS ANTIMICROBIAL AGENTS

(71) Applicant: DEINOVE, GRABELS (FR)

(72) Inventor: DOMINIQUE LE BELLER, JAUX (FR)

(21) Appl. No.: 16/475,696

(22) PCT Filed: Jan. 2, 2018
(86) PCT No.: PCT/EP2018/050073

§ 371 Date: Jul. 3, 2019

(30) Foreign Application Priority Data
Jan. 3, 2017 (EP) 17305005.5
Jan. 3, 2017 (EP) 17305006.3
Oct. 12, 2017 (EP) 17196245.9

(51) Int. Cl. C07K 7/06; A61P 31/06; A61P 31/10

(52) U.S. Cl. CPC C07K 7/06; A61P 31/10; A61P 31/06

(57) ABSTRACT

The present invention relates to novel antimicrobial compounds and their uses, in particular as medicament, disinfectant, preservative or phytosanitary agent.

### FIELD OF THE INVENTION

The present invention relates to novel antimicrobial compounds, to pharmaceutical compositions comprising said compounds and to the uses thereof, in particular as medicament, disinfectant, preservative or phytosanitary agent.

### BACKGROUND OF THE INVENTION

The evolution and spread of antibiotic resistance among bacteria is a major public health problem today, especially in the hospital setting with the emergence of multidrug resistant strains. Intensive research efforts have led to the development of new antibiotics effective against these resistant strains. Nevertheless, through use, mechanisms of resistance to these drugs emerge and limit their efficacy.

Infections caused by multidrug-resistant Gram-positive bacteria represent a major public health burden, not just in terms of morbidity and mortality, but also in terms of increased expenditure on patient management and implementation of infection control measures.

In particular, Staphylococcus aureus is one of the most commonly identified pathogens in human medicine and is a major cause of nosocomial infections and community-acquired infections. Methicillin-resistant Staphylococcus aureus (MRSA) was reported for the first time in 1961 and is now widespread in hospitals all over the world.

The increasing burden of Gram-positive infections is not limited to micro-organisms within the genus Staphylococcus, but also involves for example Enterococcus spp., in particular with the emergence of vancomycin-resistant enterococci (VRE) strains or Streptococcus spp. with reduced susceptibility to penicillins and macrolides.

Therefore, the search for new chemical entities with antimicrobial properties and structures differing from those found in conventional antibiotics is viewed as a pressing need to develop new ways to curb these resistant infections.

### SUMMARY OF THE INVENTION

The present invention relates to novel antimicrobial compounds.

In particular, the present invention relates to a compound of formula (I)

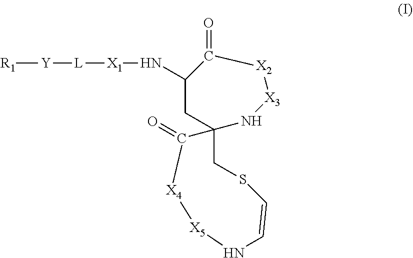

wherein

X₁, X₂, X₃, X₄ and X₅ are independently selected and each represents an amino acid,

L is a bifunctional linker, preferably selected from the group consisting of —C(═O)—, —SO₂—, —C(═S)—, —O—C(═S)—, —NHC(═S)—, —PO—, —OPO—, —OC(═O)— and —NHC(═O)—,

Y is a C₆-C₂₀ saturated or unsaturated linear hydrocarbon chain, said chain being optionally (i) interrupted by one or several heteroatoms independently selected from N, S and O, and/or (ii) interrupted by one or several groups independently selected from a phenyl group and a 5 or 6-membered-ring heterocycle, said phenyl group or heterocycle being optionally substituted by one or several groups independently selected from C₁-C₃ alkyl groups, —OH and C₁-C₃ alkoxy groups, and/or (iii) substituted by one or several groups independently selected from C₁-C₃ alkyl groups, halogens, —OH, methoxy or acetoxy, and

R₁ is selected from the group consisting of hydrogen and a basic group,

or any pharmaceutically acceptable salt, solvate or hydrate thereof.

Preferably, R₁ is selected from the group consisting of hydrogen and a basic group selected from the group consisting of —NR₂R₃,

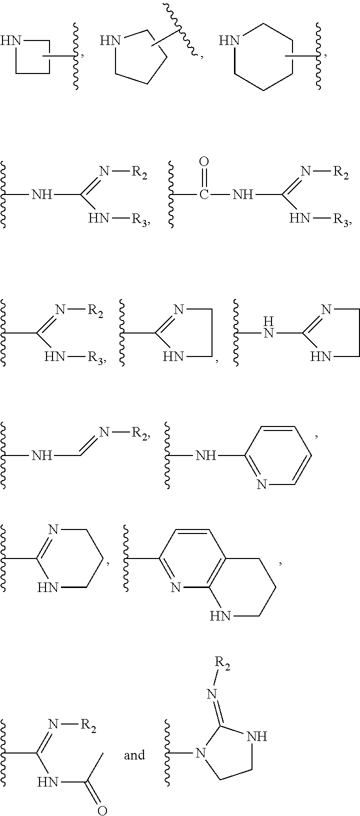

with R₂ and R₃ being independently selected from hydrogen, C₁-C₃ alkyl groups and —C(═O)R₄, and R₄ being a C₁-C₃ alkyl group.

More preferably, R₁ is

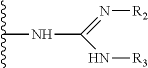

with R₂ and R₃ being independently selected from hydrogen and C₁-C₃ alkyl groups, preferably being methyl.

Preferably, L is —C(═O)—.

In some embodiments,

a) X₁ is an amino acid selected from the group consisting of A, G, Q, L, W, S and T, preferably A or G, more preferably A; and/or

b) X₂ is an amino acid selected from the group consisting of R, L, V, I, G, T, A, and S, preferably from L, V, I, G and A, even more preferably from L, V, I and A, more preferably L or I, and even more preferably I; and/or

c) X₃ is an amino acid selected from the group consisting of G, S, A, C, L, V, T, P and I, preferably from G, S, A and T, more preferably G or S, and more preferably S; and/or

d) X₄ is an amino acid selected from the group consisting of I, Q, S, N, E, D, W, H, P and T, preferably Q or N, more preferably N; and/or

e) X₅ is an amino acid selected from the group consisting of G, A, S, T, N, R, H, P and D, preferably from G, A, S and T, more preferably G or S, even more preferably G.

In some other embodiments,

X₁ is an amino acid selected from the group consisting of A and G, preferably is A, and/or

X₂ is an amino acid selected from the group consisting of L, V, I, G, A, R, T and S, preferably from the group consisting of L, V, I, G, A and T, more preferably from the group consisting of L, V, I, G and A, and even more preferably from the group consisting of L or I and/or

X₃ is an amino acid selected from the group consisting of G, A, S and T, preferably from the group consisting of G, A and S, and more preferably from the group consisting of G and S, and/or

X₄ is an amino acid selected from the group consisting of Q, N, I, S, E, D, W, H, P and T, preferably from the group consisting of Q, N, S, E and D, more preferably from the group consisting of Q and N, and/or

X₅ is an amino acid selected from the group consisting of G, A, S and T, preferably from the group consisting of G, S and T, more preferably from the group consisting of G and S.

In some further embodiments,

X₁ is an amino acid selected from the group consisting of A and G, preferably is A, and/or

X₂ is an amino acid selected from the group consisting of L, V, I, G and A, preferably from the group consisting of L or I and/or

X₃ is an amino acid selected from the group consisting of G, A, S and T, preferably from the group consisting of G and S, and/or

X₄ is an amino acid selected from the group consisting of Q and N, and/or

X₅ is an amino acid selected from the group consisting of G, A, S and T, preferably from the group consisting of G and S.

In some other embodiments,

X₁ is an amino acid selected from the group consisting of A and G, preferably is A,

X₂ is an amino acid selected from the group consisting of V, I, G, T and A,

X₃ is an amino acid selected from the group consisting of A and S,

X₄ is an amino acid selected from the group consisting of N, S, E and D, and

X₅ is an amino acid selected from the group consisting of G and T.

Preferably, X₁ is A, X₂ is L, X₃ is G, X₄ is Q and X₅ is S or X₁ is A, X₂ is I, X₃ is S, X₄ is N and X₅ is G, or X₁ is A, X₂ is V, X₃ is S, X₄ is S and X₅ is G, or X₁ is A, X₂ is T, X₃ is A, X₄ is D and X₅ is G, or X₁ is A, X₂ is T, X₃ is S, X₄ is D and X₅ is G, or X₁ is A, X₂ is A, X₃ is S, X₄ is E and X₅ is T, or X₁ is A, X₂ is G, X₃ is S, X₄ is E and X₅ is G.

Preferably, when X₁ is A, X₂ is L, X₃ is G, X₄ is Q, X₅ is S, L is —C(═O)—, and R₁ is

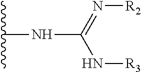

with R₂ and R₃ being methyl,

then Y is not selected from the group consisting of —(CH₂)ₘ— with m being 14 or 16, and a C₁₆ linear hydrocarbon chain comprising one unsaturation, said unsaturation being a double bond.

Preferably, Y is a C₆-C₂₀ saturated or unsaturated linear hydrocarbon chain optionally interrupted by a phenyl group. In particular, Y may be is a C₆-C₁₃ saturated or unsaturated linear hydrocarbon chain, preferably a C₉-C₁₃ saturated or unsaturated linear hydrocarbon chain.

The invention further relates to a compound of formula (V)

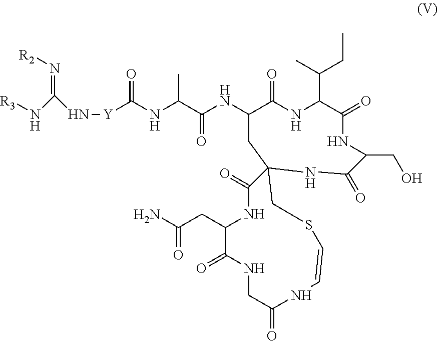

wherein

R₂ and R₃ are hydrogen or methyl and

Y is a C₆-C₁₃ saturated or unsaturated linear hydrocarbon chain, preferably selected from the group consisting of

(i) a C₁₀ saturated linear hydrocarbon chain,

(ii) C₁₀ unsaturated linear hydrocarbon chains comprising one or two double bonds, preferably selected from —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and —(CH₂)₄—(CH₂)ₘ—CH═CH—(CH₂)ₙ—, wherein m and n are independently selected from 0 and integers from 1 to 4, and m+n=4; and

(iii) C₁₂ unsaturated linear hydrocarbon chains comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2, and more preferably —(CH₂)₄—CH═CH—(CH₂)₂—CH═CH—CH═CH.

The present invention relates to a compound of the invention, in particular an antimicrobial compound of the invention or any acceptable salt, solvate or hydrate thereof, as a medicament.

The present invention also relates to a pharmaceutical composition comprising a compound of the invention, or any acceptable salt, solvate or hydrate thereof, and a pharmaceutically acceptable carrier and/or excipient.

It further relates to a compound of the invention, or any acceptable salt, solvate or hydrate thereof, for use in the treatment of a microbial infection, preferably a bacterial or fungal infection.

The microbial infection may be a bacterial infection, preferably a infection due to a Gram-positive bacterium. In particular, the Gram-positive bacterium may be selected from the group consisting of methicillin sensitive and resistant Staphylococcus aureus and Staphylococcus epidermidis, vancomycin sensitive and resistant Enterococcus faecalis and Enterococcus faecium, Bacillus subtilis, penicillin sensitive and resistant Streptococcus pneumonia, Streptococcus pyogenes, Streptococcus agalactiae, Streptococcus mitis, Streptococcus oralis, Clostridium difficile and Propionibacterium acnes.

Alternatively, the microbial infection may be due to a mycobacterium strain, preferably Mycobacterium tuberculosis or may be due to a pathogenic fungus, preferably selected from the group consisting Candida albicans, Candida parapsilosis, Candida krusei, Candida glabrata and Candida tropicalis and Cryptococcus neoformans.

The present invention also relates to a phytosanitary composition comprising a compound of the invention or any acceptable salt, solvate or hydrate thereof, and optionally an acceptable carrier and/or excipient, as well as a method for preventing or treating a plant against phytopathogens, preferably bacteria or fungi, comprising contacting said plant with an effective amount of a compound of the invention or any acceptable salt, solvate or hydrate thereof.

### BRIEF DESCRIPTION OF THE DRAWINGS

FIG. 1: LC-MS spectra—Crude extract from Nocardia altamirensis.

FIG. 2: MS-Spectra—Crude extract from Nocardia altamirensis.

FIG. 3: LC-MS analysis of the fraction containing compound NOC1 from Nocardia terpenica.

FIG. 4: MS/MS spectra of compound NOC1 from Nocardia terpenica.

FIG. 5 : LC-UV analysis of the reaction mixture after 22 hours. The core peptide corresponds to the peak of RT: 1.18 min. Compound A corresponds to the peak of RT: 6.98 min.

FIG. 6: LC-MS analysis of the reaction mixture after 22 hours. The core peptide corresponds to the peak of RT: 1.12 min. Compound A corresponds to the peak of RT: 7.05 min.

FIG. 7: MS spectra of the core peptide (RT: 1.12 min).

FIG. 8: MS/MS spectra of the core peptide (RT: 1.12 min).

FIG. 9: MS spectra of the reacylated core peptide.

FIG. 10: ¹H NMR spectra of compound Noc1 in CD₃CN:D₂O 60:40.

FIG. 11: COSY ¹H-¹H NMR spectra of compound Noc1 in CD₃CN:D₂O 60:40.

FIG. 12: HSQCY ¹H-¹³C RMN spectra of compound Noc1 in CD₃CN:D₂O 60:40.

FIG. 13: Intra-residual fatty acid chain NMR assignment of compound Noc1.

### DETAILED DESCRIPTION OF THE INVENTION

The present invention relates to a new class of antimicrobial compounds. These compounds typically comprise a core polycyclic peptide and a fatty acid moiety. Based on their structure, these new compounds have been herein referred to as lipolanthipeptides, polyclic RiPPs (ribosomally synthesized post-transcriptionally modified peptides) with a fatty acid substituent. Such compounds exhibit potent antimicrobial activity, particularly against Gram positive bacteria, including vancomycin-resistant Enterococcus strains or methicillin-resistant Staphylococcus strains, as well as against mycobacteria and pathogenic fungi such as Candida strains.

Accordingly, in a first aspect, the present invention relates to compounds, in particular antimicrobial compounds, comprising a bicyclic core peptide and a lipophilic moiety. The compounds of the invention are of formula (I):

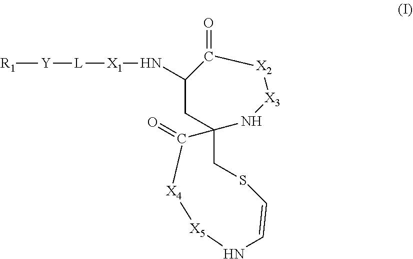

wherein

X₁, X₂, X₃, X₄ and X₅ are independently selected and each represents an amino acid,

L is a bifunctional linker, preferably selected from the group consisting of —C(═O)—, —SO₂—, —C(═S)—, —OC(═S)—, —PO—, —OPO—, —OC(═O)—, —NHC(═O)— and —NHC(═S),

Y is a saturated or unsaturated linear hydrocarbon chain, optionally substituted and/or interrupted, preferably a C₆-C₂₀ saturated or unsaturated linear hydrocarbon chain, said chain being optionally (i) interrupted by one or several heteroatoms independently selected from N, S and O, and/or (ii) interrupted by one or several groups independently selected from a phenyl group and a 5 or 6-membered-ring heterocycle, said phenyl group or heterocycle being optionally substituted, for example, by one or several groups independently selected from C₁-C₃ alkyl groups, —OH and C₁-C₃ alkoxy groups, and/or (iii) substituted by one or several groups independently selected from C₁-C₃ alkyl groups, halogens, —OH, methoxy or acetoxy, and

R₁ is selected from the group consisting of hydrogen and a basic group,

or any pharmaceutically acceptable salt or hydrate thereof.

As used herein, the term “amino acid” or “amino acid residue” refers to any of the naturally occurring amino acids, including rare amino acids, as well as non-natural analogues.

In preferred embodiments, the term “amino acid” refers to any of the 20 naturally occurring amino acids which may be represented by their one-letter code according to the following nomenclature: A: alanine, C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan and Y: tyrosine. In some embodiments, the side chains of these amino acid residues may be chemically modified, for example by glycosylation, amidation, acylation, acetylation or methylation.

The amino acids may be in the L or D configuration, or a combination of both. In preferred embodiments, X₁, X₂, X₃, X₄ and X₅ represent amino acids in the L configuration.

The amino acid residues may be linked to the adjacent components through “classical” CONH peptide bonds or through pseudo-peptide bonds. In particular, the compound of the invention may comprise one or several pseudo-peptide bonds replacing one or several CONH peptide bonds.

In preferred embodiments, X₁, X₂, X₃, X₄ and X₅ are linked to the adjacent components through “classical” CONH peptide bonds and the compound of the invention is of formula (II)

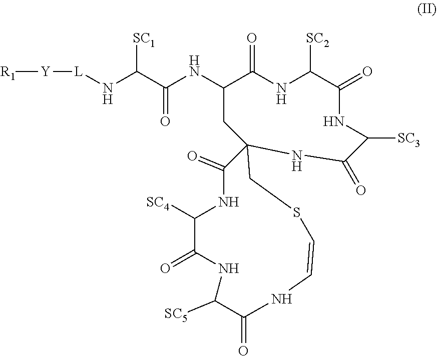

wherein

R¹, Y and L have the same meaning as described above and

SC₁, SC₂, SC₃, SC₄ and SC₅ represent the side-chains of the amino acids X₁, X₂, X₃, X₄ and X₅, respectively.

The compound of formula (I) or (II) has preferably one or several of the following features:

a) X₁ is an amino acid selected from the group consisting of A, G, Q, L, W, S and T, preferably A or G, more preferably A; and/or

b) X₂ is an amino acid selected from the group consisting of R, L, V, I, G, T, A, and S, preferably from L, V, I, G and A, even more preferably from L, V, I and A, more preferably L or I, and even more preferably I; and/or

c) X₃ is an amino acid selected from the group consisting of G, S, A, C, L, V, T, P and I, preferably from G, S, A and T, more preferably G or S, and more preferably S; and/or

d) X₄ is an amino acid selected from the group consisting of I, Q, S, N, E, D, W, H, P and T, preferably Q or N, more preferably N; and/or

e) X₅ is an amino acid selected from the group consisting of G, A, S, T, N, R, H, P and D, preferably from G, A, S and T, more preferably G or S, even more preferably G.

More preferably, the compound of formula (I) or (II) may have one or several of the following features:

a) X₁ is an amino acid selected from the group consisting of A and G, preferably is A, and/or

b) X₂ is an amino acid selected from the group consisting of L, V, I, G, A, R, T and S, preferably from the group consisting of L, V, I, G, A and T, more preferably from the group consisting of L, V, I, G and A, and even more preferably from the group consisting of L or I and/or

c) X₃ is an amino acid selected from the group consisting of G, A, S and T, preferably from the group consisting of G, A and S, and more preferably from the group consisting of G and S, and/or

d) X₄ is an amino acid selected from the group consisting of Q, N, I, S, E, D, W, H, P and T, preferably from the group consisting of Q, N, S, E and D, more preferably from the group consisting of Q and N, and/or

e) X₅ is an amino acid selected from the group consisting of G, A, S and T, preferably from the group consisting of G, S and T, more preferably from the group consisting of G and S.

Alternatively, the compound of formula (I) or (II) may have one or several of the following features:

a) X₁ is an amino acid selected from the group consisting of A and G, preferably is A, and/or

b) X₂ is an amino acid selected from the group consisting of L, V, I, G, A, R, T and S, preferably from the group consisting of V, T, A and G, and/or

c) X₃ is an amino acid selected from the group consisting of G, A, S and T, preferably from the group consisting of A and S, and/or

d) X₄ is an amino acid selected from the group consisting of Q, N, I, S, E, D, W, H, P and T, preferably from the group consisting of S, E and D, and/or

e) X₅ is an amino acid selected from the group consisting of G, A, S and T, preferably from the group consisting of G and T.

In a particular embodiment, the compound of formula (I) or (II) has one or several of the following features:

a) X₁ is an amino acid selected from the group consisting of A and G, preferably is A, and/or

b) X₂ is an amino acid selected from the group consisting V, T, A and G, and/or

c) X₃ is an amino acid selected from the group consisting of A and S, and/or

d) X₄ is an amino acid selected from the group consisting of S, E and D, and/or

e) X₅ is an amino acid selected from the group consisting of G and T.

Even more preferably, the compound of formula (I) or (II) has preferably one or several of the following features:

a) X₁ is an amino acid selected from the group consisting of A or G, preferably is A; and/or

b) X₂ is an amino acid selected from the group consisting of L, V, I, G and A, preferably an amino acid selected from the group consisting of L, V, I and A, more preferably is selected from the group consisting of L and I, and even more preferably is I; and/or

c) X₃ is an amino acid selected from the group consisting of G, A, S and T, preferably an amino acid selected from the group consisting of G and S, and more preferably is S; and/or

d) X₄ is an amino acid selected from the group consisting of Q and N, preferably is N; and/or

e) X₅ is an amino acid selected from the group consisting of G, A, S and T, preferably is an amino acid selected from the group consisting of G and S, and more preferably is G.

The compound of formula (I) or (II) may meet one feature, two features [for instance a) and b); a) and c); a) and d); a) and e); b) and c); b) and d); b) and e); c) and d); c) and e); d) and e)], three features [for instance a), b) and c); a), b) and d); a), b) and e); a), c) and d); a), c) and e); a), d) and e); b), c) and d); b), c) and e); c), d) and e)], four features [a), b), c) and d); a), b), c) and e); a), b), d) and e); a), c), d) and e); b), c), d) and e)], or five features [i.e. a), b), c), d) and e)] as described above.

In a particular embodiment,

X₁ is an amino acid selected from the group consisting of A and G,

X₂ is an amino acid selected from the group consisting of L, V, I, G and A,

X₃ is an amino acid selected from the group consisting of G, A, S and T,

X₄ is an amino acid selected from the group consisting of Q, I and N, and

X₅ is an amino acid selected from the group consisting of G, A, S and T.

In a more particular embodiment,

X₁ is an amino acid selected from the group consisting of A and G, preferably is A,

X₂ is an amino acid selected from the group consisting of L, V and I, preferably selected from the group consisting of L and I,

X₃ is an amino acid selected from the group consisting of G and S,

X₄ is an amino acid selected from the group consisting of Q, I and N, preferably is Q or N, and

X₅ is an amino acid selected from the group consisting of G and S, preferably is G.

In another particular embodiment,

X₁ is an amino acid selected from the group consisting of A and G,

X₂ is an amino acid selected from the group consisting of L, V, I, G and A,

X₃ is an amino acid selected from the group consisting of G, A, S and T,

X₄ is an amino acid selected from the group consisting of Q and N, and

X₅ is an amino acid selected from the group consisting of G, A, S and T.

In a more particular embodiment,

X₁ is an amino acid selected from the group consisting of A and G, preferably is A,

X₂ is an amino acid selected from the group consisting of L, V and I, preferably selected from the group consisting of L and I, more preferably is I,

X₃ is an amino acid selected from the group consisting of G and S, preferably is S,

X₄ is an amino acid selected from the group consisting of Q and N, preferably is N, and

X₅ is an amino acid selected from the group consisting of G and S, preferably is G.

In a particular embodiment,

X₁ is an amino acid selected from the group consisting of A and G, preferably is A,

X₂ is an amino acid selected from the group consisting of L, V, I, G, T and A,

X₃ is an amino acid selected from the group consisting of G, A and S,

X₄ is an amino acid selected from the group consisting of Q, N, S, E and D, and

X₅ is an amino acid selected from the group consisting of G, S and T.

In another particular embodiment,

X₁ is an amino acid selected from the group consisting of A and G, preferably is A,

X₂ is an amino acid selected from the group consisting of V, G, T and A,

X₃ is an amino acid selected from the group consisting of A and S,

X₄ is an amino acid selected from the group consisting of S, E and D, and

X₅ is an amino acid selected from the group consisting of G and T.

In another particular embodiment,

X₁ is an amino acid selected from the group consisting of A and G, preferably is A,

X₃ is an amino acid selected from the group consisting of S and G, and

X₅ is an amino acid selected from the group consisting of S and G.

Optionally, X₂ is an amino acid selected from the group consisting of L, V, I, G, T and A, and/or X₄ is an amino acid selected from the group consisting of Q, N, S, E and D.

In another particular embodiment,

X₁ is A,

X₂ is an amino acid selected from the group consisting of G, T and A, preferably G and T,

X₃ is an amino acid selected from the group consisting of S and A, preferably is A,

X₄ is an amino acid selected from the group consisting of D and E, and

X₅ is an amino acid selected from the group consisting of T and G, preferably is T.

In another particular embodiment,

X₁ is an amino acid selected from the group consisting of A and G, preferably is A,

X₂ is an amino acid selected from the group consisting of V, I, G, T and A,

X₃ is an amino acid selected from the group consisting of A and S,

X₄ is an amino acid selected from the group consisting of N, S, E and D, and

X₅ is an amino acid selected from the group consisting of G and T.

In a further particular embodiment, X₂ is L and X₃ is G.

In another particular embodiment, X₃ is G and X₄ is I or Q.

In another particular embodiment, X₃ is G and X₄ is Q.

In a further particular embodiment, X₄ is Q and X₅ is S.

In another particular embodiment, X₂ is L, X₃ is G and X₄ is I.

In another particular embodiment, X₂ is L, X₃ is G and X₄ is Q.

In another particular embodiment, X₁ is A, X₂ is L and, X₃ is G.

In another particular embodiment, X₁ is A, X₃ is G and X₄ is Q.

In another particular embodiment, X₁ is A, X₂ is T, X₄ is D and X₅ is G.

In another particular embodiment, X₁ is A and X₃ is S.

In another particular embodiment, X₁ is A and X₅ is G.

In another particular embodiment, X₁ is A, X₃ is S and X₅ is G. Optionally, in this embodiment, X₂ is an amino acid selected from the group consisting of L, V, I, G, T and A, and/or X₄ is an amino acid selected from the group consisting of Q, N, S, E and D.

In a preferred embodiment, X₁ is A, X₂ is L, X₃ is G, X₄ is Q and X₅ is S.

In another preferred embodiment, X₁ is A, X₂ is I, X₃ is S, X₄ is N and X₅ is G.

In another preferred embodiment, X₁ is A, X₂ is V, X₃ is S, X₄ is S and X₅ is G.

In another preferred embodiment, X₁ is A, X₂ is T, X₃ is A, X₄ is D and X₅ is G.

In another preferred embodiment, X₁ is A, X₂ is T, X₃ is S, X₄ is D and X₅ is G.

In another preferred embodiment, X₁ is A, X₂ is A, X₃ is S, X₄ is E and X₅ is T.

In another preferred embodiment, X₁ is A, X₂ is G, X₃ is S, X₄ is E and X₅ is G.

Preferably, in all embodiments described herein, X₁ is A.

The bicyclic core peptide and the lipophilic moiety are linked via a bifunctional linker. As used herein, the term “bifunctional linker” refers to any chemical group being able to connect two chemical groups, and in particular being able to covalently connect at the same time (i) a hydrocarbon chain and (ii) an amino group.

Typically, L comprises 1 to 25 atoms, preferably 1 to 10 atoms, and at least one heteroatom selected from O, S and P.

Preferably, L is selected from the group consisting of—C(═O)—, —SO₂—, —C(═S)—, —O—C(═S)—, —NHC(═S)—, —PO—, —OPO—, —OC(═O)— and —NHC(═O)—, more preferably from the group consisting of —C(═O)—, —SO₂—, —C(═S)—, —OC(═O)— and —NHC(═O)—.

In preferred embodiments, L is —C(═O)—.

In a preferred embodiment, the compounds are of formula (III):

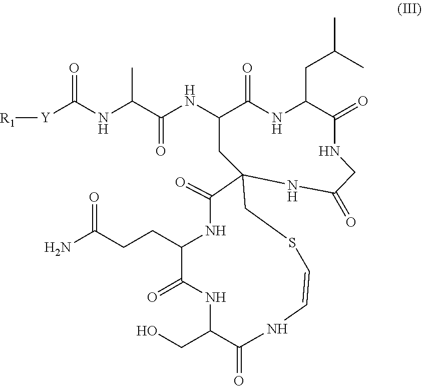

wherein Y and R₁ are as defined above and hereafter.

In another preferred embodiment, the compounds are of formula (IV):

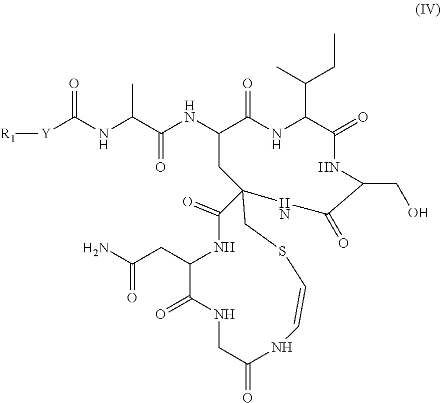

wherein Y and R₁ are as defined above and hereafter.

The compounds of the invention comprise a lipophilic moiety Y. Preferably, Y is a saturated or unsaturated linear hydrocarbon chain, preferably a C₆-C₂₀ saturated or unsaturated linear hydrocarbon chain, optionally substituted and/or interrupted.

This hydrocarbon chain may optionally be

(i) interrupted by one or several heteroatoms independently selected from N, S and O, and/or

(ii) interrupted by one or several groups independently selected from a phenyl group and a 5 or 6-membered-ring heterocycle, said phenyl group or heterocycle being optionally substituted, for example, by one or several groups independently selected from C₁-C₃ alkyl groups, —OH and C₁-C₃ alkoxy groups, and/or

(iii) substituted by one or several groups independently selected from C₁-C₃ alkyl groups, halogens, —OH, methoxy and acetoxy.

By “C₆-C₂₀ saturated linear hydrocarbon chain” is meant a linear hydrocarbon chain having from 6 to 20 carbons, i.e. 6, 7, 8, 9, 10, 11, 12, 13, 14, 15, 16, 17, 18, 19 or 20 carbons, and which does not comprise any unsaturation i.e. any double nor triple bonds.

By “C₆-C₂₀ unsaturated linear hydrocarbon chain” is meant a linear hydrocarbon chain having from 6 to 20 carbons and which comprises at least one unsaturation i.e. at least one double bond and/or at least one triple bond, preferably at least one double bond.

In the case of an unsaturated linear hydrocarbon chain comprising several unsaturations, each unsaturation may be a triple bond or a double bond. Preferably each unsaturation is a double bond. The double bonds may have indifferently trans configuration (E) or cis configuration (Z). Preferably, the double bond(s) is/are in cis configuration.

Preferably, in embodiments wherein Y is an unsaturated hydrocarbon chain, said chain comprises from 1 to 4 double bonds, more preferably from 1 to 3 double bonds, and even more preferably one or two double bonds.

In particular, Y may be an unsaturated linear hydrocarbon chain comprising one, two or three double bonds. Preferably, Y is selected from the group consisting of

(i) —(CH₂)₄—(CH₂)ₘ—CH═CH—(CH₂)ₙ—CH═CH—(CH₂)ₚ—CH═CH—(CH₂)_{q}—,

wherein m, n, p and q are independently selected from 0 and integers from 1 to 10, and 0≤m+n+p+q≤10, preferably from 0 and integers from 1 to 6, and 0≤m+n+p+q≤6, and more preferably from 0 and integers from 1 to 3, and 0≤m+n+p+q≤3;

(ii) —(CH₂)₄—(CH₂)ₘ—CH═CH—(CH₂)ₙ—CH═CH—(CH₂)ₚ—,

wherein m, n and p are independently selected from 0 and integers from 1 to 12, and 0≤m+n+p≤12, preferably from 0 and integers from 1 to 8, and 0≤m+n+p≤8, and more preferably from 0 and integers from 1 to 5, and 0≤m+n+p≤5; or

(iii) —(CH₂)₄—(CH₂)ₘ—CH═CH—(CH₂)ₙ—,

wherein m and n are independently selected from 0 and integers from 1 to 14, and 0≤m+n≤14, preferably from 0 and integers from 1 to 10, and 0≤m+n≤10, more preferably from 0 and integers from 1 to 7, and 0≤m+n≤7.

In an embodiment, Y is a C₆-C₁₈ saturated or unsaturated linear hydrocarbon chain, preferably a C₁₅-C₁₈ saturated or unsaturated linear hydrocarbon chain, i.e. a C₁₅, C₁₆, C₁₇ or C₁₈ saturated or unsaturated linear hydrocarbon chain, more preferably a C₁₅-C₁₈ saturated linear hydrocarbon chain.

In another embodiment, Y is a C₆-C₁₆ saturated or unsaturated linear hydrocarbon chain, preferably a C₉-C₁₆ saturated or unsaturated linear hydrocarbon chain.

In a particular embodiment, Y is a C₁₄-C₁₆ saturated or unsaturated linear hydrocarbon chain, preferably a C₁₄ or C₁₆ saturated or unsaturated linear hydrocarbon chain.

Preferably, Y is selected from the group consisting of C₁₆ and C₁₄ saturated linear hydrocarbon chains and C₁₆ and C₁₄ unsaturated linear hydrocarbon chains comprising one, two or three double bonds, preferably one double bond.

In particular, the C₁₆ and C₁₄ unsaturated linear hydrocarbon chains comprising one, two or three double bonds may be selected from the group consisting of:

(i) —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 6, and m+n+p+q=4 or 6, preferably 2≤q;

(ii) —(CH₂)₄—(CH₂)ₘ—CH═CH—(CH₂)ₙ—CH═CH—(CH₂)ₚ—, wherein m, n and p are independently selected from 0 and integers from 1 to 8, and m+n+p=6 or 8, preferably 2≤p; and

(iii) —(CH₂)₄—(CH₂)ₘ—CH═CH—(CH₂)ₙ—, wherein m and n are independently selected from 0 and integers from 1 to 10, and m+n=8 or 10, preferably 2≤n.

More preferably, Y is selected from the group consisting of C₁₆ and C₁₄ saturated linear hydrocarbon chains and C₁₆ unsaturated linear hydrocarbon chains comprising one double bond, preferably —(CH₂)₄—(CH₂)ₘ—CH═CH—(CH₂)ₙ—, wherein m and n are independently selected from 0 and integers from 1 to 10, and m+n=10, preferably 2≤n.

In another embodiment, Y is a C₆-C₁₃ saturated or unsaturated linear hydrocarbon chain, preferably a C₉-C₁₃ saturated or unsaturated linear hydrocarbon chain.

In a particular embodiment, Y is a C₁₀-C₁₂ saturated or unsaturated linear hydrocarbon chain, preferably a C₁₀ or C₁₂ saturated or unsaturated linear hydrocarbon chain.

Preferably, Y is selected from the group consisting of C₁₀ and C₁₂ saturated linear hydrocarbon chains and C₁₀ and C₁₂ unsaturated linear hydrocarbon chains comprising one, two or three double bonds.

In particular the C₁₀ and C₁₂ unsaturated linear hydrocarbon chains comprising one, two or three double bonds may be selected from the group consisting of:

(i) —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=0 or 2;

(ii) —(CH₂)₄—(CH₂)ₘ—CH═CH—(CH₂)ₙ—CH═CH—(CH₂)ₚ—, wherein m, n and p are independently selected from 0 and integers from 1 to 4, and m+n+p=2 or 4; and

(iii) —(CH₂)₄—(CH₂)ₘ—CH═CH—(CH₂)ₙ—, wherein m and n are independently selected from 0 and integers from 1 to 6, and m+n=4 or 6.

More preferably, Y is selected from the group consisting of

(i) a C₁₀ saturated linear hydrocarbon chain,

(ii) C₁₀ unsaturated linear hydrocarbon chains comprising one or two double bonds, preferably selected from:

—(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and —(CH₂)₄—(CH₂)ₘ—CH═CH—(CH₂)ₙ—, wherein m and n are independently selected from 0 and integers from 1 to 4, and m+n=4; and

(iii) C₁₂ unsaturated linear hydrocarbon chains comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2.

In a particular embodiment, Y is selected from the group consisting of C₁₀, C₁₂, C₁₄ and C₁₆ saturated or unsaturated linear hydrocarbon chains.

In a more particular embodiment, Y is selected from the group consisting of C₁₀, C₁₄ and C₁₆ saturated linear hydrocarbon chains, C₁₆ unsaturated linear hydrocarbon chains comprising one double bond, C₁₀ unsaturated linear hydrocarbon chains comprising one or two double bonds, and C₁₂ unsaturated linear hydrocarbon chains comprising three double bonds. Preferably, unsaturated chains are as defined above.

Preferably, in embodiments wherein Y is a C₁₂ unsaturated linear hydrocarbon chain comprising three double bonds, Y is —(CH₂)₄—CH═CH—(CH₂)₂—CH═CH—CH═CH—.

As mentioned hereabove, the saturated or unsaturated linear hydrocarbon chain may be substituted by one or several groups independently selected from C₁-C₃ alkyl groups, halogens, —OH, methoxy and acetoxy.

As used herein, C₁-C₃ alkyl groups encompass methyl, ethyl, propyl and isopropyl. Halogens may be selected from F, Cl and Br.

In a particular embodiment, the saturated or unsaturated linear hydrocarbon chain is substituted by one group selected from C₁-C₃ alkyl groups, halogens, —OH, methoxy and acetoxy, preferably —OH.

The substituted saturated or unsaturated linear hydrocarbon chain may be any saturated or unsaturated linear hydrocarbon chain as described above.

In preferred embodiments, the saturated or unsaturated linear hydrocarbon chain is unsubstituted.

In some embodiments, the hydrocarbon chain as described above may be interrupted by one or several heteroatoms independently selected, preferably from N, S and O, and/or by one or several groups independently selected from a phenyl group and a 5 or 6-membered-ring heterocycle, said phenyl group or heterocycle being optionally substituted, for example, by one or several groups independently selected from C₁-C₃ alkyl groups, —OH and C₁-C₃ alkoxy groups.

As used herein, the term “heteroatom” refers to any atom that is not carbon or hydrogen. In preferred embodiments, this term refers to N, S, or O.

The term “heterocycle”, as used herein, refers to 5- or 6-membered heterocyclic ring systems comprising one or more heteroatoms, preferably 1 or 2 endocyclic heteroatoms. Preferably, they are monocyclic systems. They may be aromatic or not. Examples of 5- or 6-membered-ring heterocycles include furan, pyrrole, thiophene, oxazole, isoxazole, thiazole, isothiazole, imidazole, pyrazole, triazole, pyridine, pyrane, piperidine, dioxane, pyrazine and pyrimidine.

In a particular embodiment, the hydrocarbon chain as described above is interrupted by one or several heterocycles, preferably by one, two or three heterocycles. In such embodiment, the heterocycle(s) may be inserted in the chain in one of the following configurations:

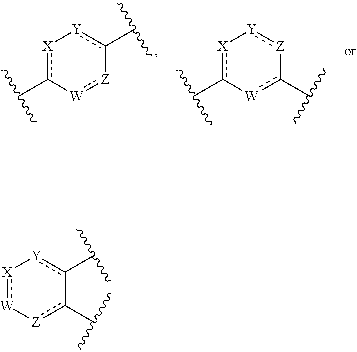

wherein X, Y, W and Z are independently selected from carbon and nitrogen,

and

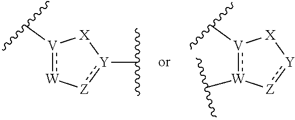

wherein X, W and Z are independently selected from carbon, nitrogen, sulfur and oxygen, and V and Y are independently selected from carbon and nitrogen,

with the proviso that the 5-membered heterocycle is inserted in the chain in one of the following configurations:

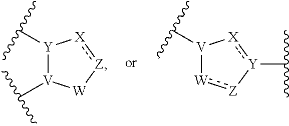

when V is nitrogen.

In embodiments wherein the chain is interrupted by several heterocycles, the configuration of each heterocycle may be independently selected from these configurations.

In another particular embodiment, the hydrocarbon chain as described above is interrupted by one or several phenyl groups, preferably by one, two or three phenyl groups. In such embodiment, phenyl groups may be inserted in the chain in one of the following configurations:

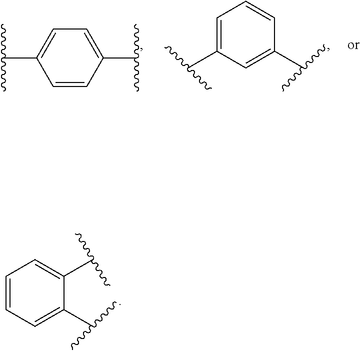

In embodiments wherein the chain is interrupted by several phenyl groups, the configuration of each phenyl group may be independently selected from these configurations.

In another embodiment, the chain is interrupted by one or two phenyl groups and one or two heterocycles.

The phenyl groups and/or heterocycles may be jointed, so as to form for example naphthalene, benzofuran, indole and/or quinoline groups, or separated by one or several carbons of the hydrocarbon chain.

The interrupted saturated or unsaturated linear hydrocarbon chain may be any saturated or unsaturated linear hydrocarbon chain as described above, including substituted and unsubstituted saturated or unsaturated linear hydrocarbon chain.

In preferred embodiments, the saturated or unsaturated linear hydrocarbon chain is not interrupted.

In most preferred embodiment, the saturated or unsaturated linear hydrocarbon chain is neither substituted nor interrupted.

In the compounds of the invention, R₁ is selected from the group consisting of hydrogen and a basic group.

As used herein, the term “basic group” refers to an organic group which is a proton acceptor. Illustrative basic groups are primary, secondary, tertiary acyclic or cyclic amines, amidines, guanidines.

In an embodiment, R1 is selected from the group consisting of hydrogen and a basic group selected from the group consisting of —NR₂R₃,

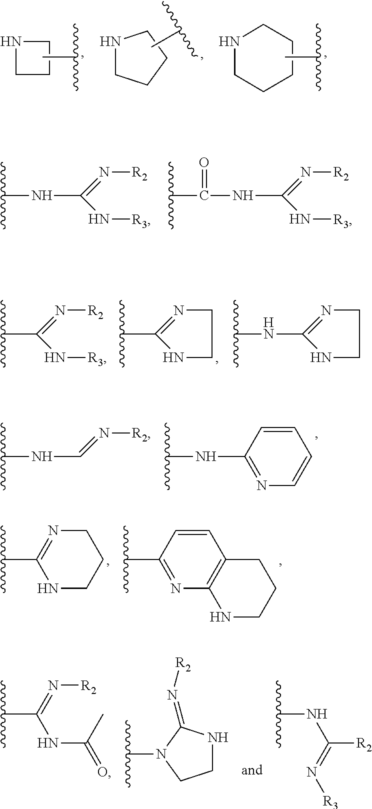

with R₂ and R₃ being independently selected from hydrogen, C₁-C₃ alkyl groups and —C(═O)R₄, and R₄ being a C₁-C₃ alkyl group.

In a more particular embodiment, R1 is selected from the group consisting of hydrogen and a basic group selected from the group consisting of —NR₂R₃,

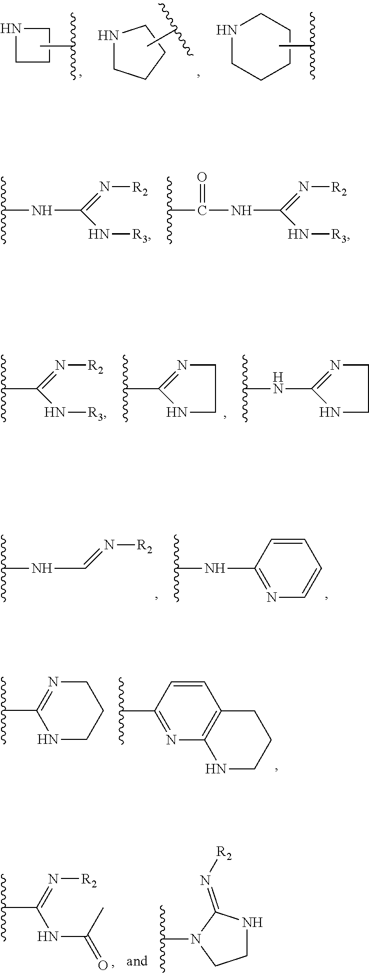

with R₂ and R₃ being independently selected from hydrogen, C₁-C₃ alkyl groups and —C(═O)R₄, and R₄ being a C₁-C₃ alkyl group.

It should be noted that tautomeric forms of the groups described above are also contemplated. As illustration, as used herein,

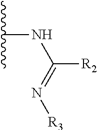

also encompasses

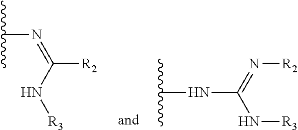

also encompasses

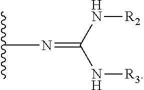

In preferred embodiments, R₁ is

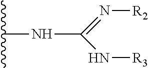

with R₂ and R₃ being independently selected from hydrogen and C₁-C₃ alkyl groups, preferably selected from hydrogen and methyl.

In particular,

R₂ and R₃ may be hydrogen,

R₂ may be hydrogen and R₃ methyl and vice-versa, or

R₂ and R₃ may be methyl.

In a preferred embodiment, R₂ and R₃ are methyl.

In a particular embodiment, the antimicrobial compound of the invention is a compound of formula (V)

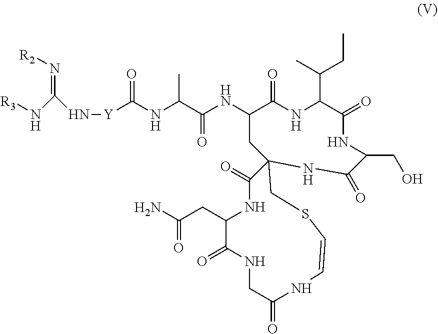

wherein Y, R₂ and R₃ are as defined above.

Preferably, Y is a C₉-C₁₃ saturated or unsaturated linear hydrocarbon chain, and R₂ and R₃ are hydrogen or methyl.

More preferably, R₂ and R₃ are hydrogen or methyl and Y is selected from the group consisting of

(i) a C₁₀ saturated linear hydrocarbon chain,

(ii) a C₁₀ unsaturated linear hydrocarbon chains comprising one or two double bonds, preferably selected from —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and —(CH₂)₄—(CH₂)ₘ—CH═CH—(CH₂)ₙ—, wherein m and n are independently selected from 0 and integers from 1 to 4, and m+n=4; and

(iii) a C₁₂ unsaturated linear hydrocarbon chains comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2, and more preferably —(CH₂)₄—CH═CH—(CH₂)₂—CH═CH—CH═CH.

Preferably, said hydrocarbon chains are unsubstituted and uninterrupted.

In a particular embodiment, the antimicrobial compound of the invention is selected from the compounds of formula (V) wherein
Y is a C₁₀ saturated linear hydrocarbon chain, and the guanidine group is monomethylated, i.e. R₂ is hydrogen and R₃ is methyl, or vice versa (compound NOC 7 of example 1);
Y is a C₁₀ saturated linear hydrocarbon chain, and the guanidine group is dimethylated, i.e. R₂ and R₃ are methyl (compound NOC 8 of example 1);
Y is a C₁₀ unsaturated linear hydrocarbon chains comprising one double bond, preferably —(CH₂)₄—(CH₂)ₘ—CH═CH—(CH₂)ₙ—, wherein m and n are independently selected from 0 and integers from 1 to 4, and m+n=4, and the guanidine group is monomethylated, i.e. R₂ is hydrogen and R₃ is methyl, or vice versa (compound NOC 4 of example 1);
Y is a C₁₀ unsaturated linear hydrocarbon chains comprising one double bond, preferably —(CH₂)₄—(CH₂)ₘ—CH═CH—(CH₂)ₙ—, wherein m and n are independently selected from 0 and integers from 1 to 4, and m+n=4, and the guanidine group is dimethylated, i.e. R₂ and R₃ are methyl (compound NOC 6 of example 1);
Y is a C₁₀ unsaturated linear hydrocarbon chains comprising two double bonds, preferably —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and the guanidine group is unmethylated, i.e. R₂ and R₃ are hydrogen (compound NOC 2 of example 1);
Y is a C₁₀ unsaturated linear hydrocarbon chains comprising two double bonds, preferably —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and the guanidine group is monomethylated, i.e. R₂ is hydrogen and R₃ is methyl, or vice versa (compound NOC 3 of example 1);
Y is a C₁₀ unsaturated linear hydrocarbon chains comprising two double bonds, preferably —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and the guanidine group is dimethylated, i.e. R₂ and R₃ are methyl (compound NOC 5 of example 1);
Y is a C₁₂ unsaturated linear hydrocarbon chain comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2, and more preferably —(CH₂)₄—CH═CH—(CH₂)₂—CH═CH—CH═CH—, and the guanidine group is monomethylated, i.e. R₂ is hydrogen and R₃ is methyl, or vice versa (compound NOC 9 of example 1); and
Y is a C₁₂ unsaturated linear hydrocarbon chain comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2, and more preferably —(CH₂)₄—CH═CH—(CH₂)₂—CH═CH—CH═CH—, and the guanidine group is dimethylated, i.e. R₂ and R₃ are methyl (compounds NOC 10 and NOC1 of example 1),

and a mixture thereof. Preferably, said hydrocarbon chains are unsubstituted and uninterrupted.

In a preferred embodiment, the antimicrobial compound of the invention is a compound of formula (VI)

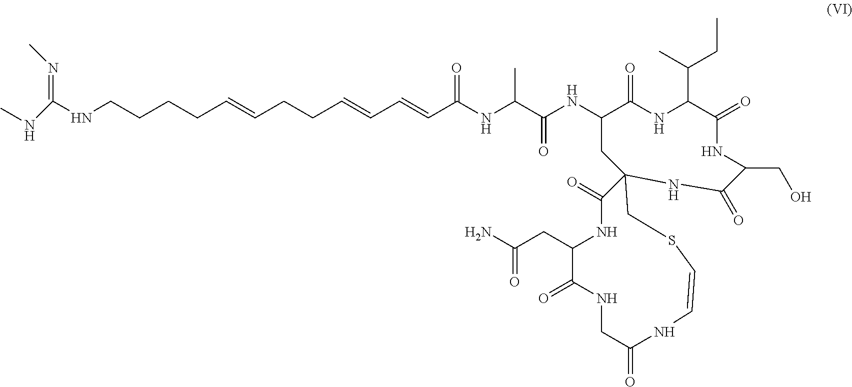 [SMILES: CCC(C)C1NC(=O)C(NC(=O)C(C)NC(=O)/C=C/C=C/CC/C=C/CCCCN/C(=N/C)NC)CC2(CS/C=C\NC(=O)CNC(=O)C(CC(N)=O)NC2=O)NC(=O)C(CO)NC1=O]

(compound NOC 1 of example 1)

In another particular embodiment, the antimicrobial compound of the invention is a compound of formula (VII)

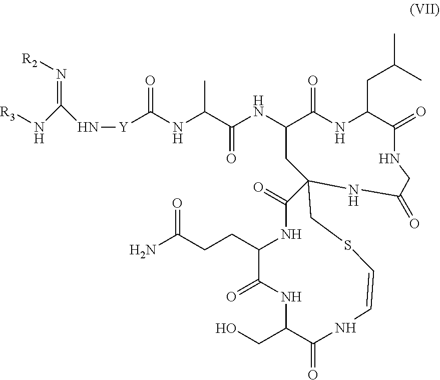

wherein Y, R₂ and R₃ are as defined above.

Preferably, Y is an unsubstituted and uninterrupted hydrocarbon chain, and R₂ and R₃ are hydrogen or methyl.

More preferably, R₂ and R₃ are hydrogen or methyl, and Y is a hydrocarbon chain selected from the group consisting of

(i) C₁₀, C₁₄ and C₁₆ saturated linear hydrocarbon chains,

(ii) C₁₆ unsaturated linear hydrocarbon chains comprising one double bond, preferably —(CH₂)₄—(CH₂)ₘ—CH═CH—(CH₂)ₙ—, wherein m and n are independently selected from 0 and integers from 1 to 10, and m+n=10;

(iii) C₁₀ unsaturated linear hydrocarbon chains comprising one or two double bonds, preferably selected from —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and —(CH₂)₄—(CH₂)ₘ—CH═CH—(CH₂)ₙ—, wherein m and n are independently selected from 0 and integers from 1 to 4, and m+n=4; and

(iv) C₁₂ unsaturated linear hydrocarbon chains comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2, and more preferably —(CH₂)₄—CH═CH—(CH₂)₂—CH═CH—CH═CH—.

Preferably, said hydrocarbon chains are unsubstituted and uninterrupted.

Even more preferably, R₂ and R₃ are hydrogen or methyl, and Y is selected from the group consisting of C₁₀ saturated linear hydrocarbon chain, C₁₀ unsaturated linear hydrocarbon chains comprising one or two double bonds, and C₁₂ unsaturated linear hydrocarbon chains comprising three double bonds, preferably —(CH₂)₄—CH═CH—(CH₂)₂—CH═CH—CH═CH.

In a particular embodiment, when X₁ is A, X₂ is L, X₃ is G, X₄ is Q, X₅ is S, L is —C(═O)—, and R₁ is

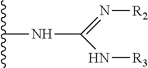

with R₂ and R₃ being methyl,

then Y is not selected from the group consisting of —(CH₂)ₘ— with m being 14 or 16, and a C₁₆ linear hydrocarbon chain comprising one unsaturation, said unsaturation being a double bond in trans conformation.

In another particular embodiment, when X₁ is A, X₂ is L, X₃ is G, X₄ is Q, X₅ is S, L is —C(═O)—, and R₁ is

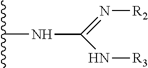

with R₂ and R₃ being methyl,

then Y is not selected from the group consisting of —(CH₂)ₘ— with m being 14 or 16, and a C₁₆ linear hydrocarbon chain comprising one unsaturation, said unsaturation being a double bond.

The present invention also relates to the pharmaceutical salts, solvates and hydrates of the compounds of the invention.

As used herein, the term “pharmaceutically acceptable salt” refers to salts which are typically non-toxic for a patient and suitable for maintaining the stability of a therapeutic agent and allowing the delivery of said agent to target cells or tissue. Pharmaceutically acceptable salts are well known in the art and may, for example, be salts of pharmaceutically acceptable mineral acids such as hydrochloric acid, hydrobromic acid, sulphuric acid and phosphoric acid; salts of pharmaceutically acceptable organic acids such as acetic acid, citric acid, maleic acid, malic acid, succinic acid, ascorbic acid and tartaric acid; salts of pharmaceutically acceptable mineral bases such as salts of sodium, potassium, calcium, magnesium or ammonium; or salts of organic bases which contain a salifiable nitrogen, commonly used in pharmaceutical technique. The methods for preparing said salts are well known to one of skill in the art.

As used herein, the term “solvate” refers to a solvent addition form that contains either stoichiometric or non stoichiometric amounts of solvent. Some compounds have a tendency to trap a fixed molar ratio of solvent molecules in the crystalline solid state, thus forming a solvate. If the solvent is water, the solvate formed is a hydrate. Hydrates are formed by the combination of one or more molecules of water with one of the substances in which the water retains its molecular state as H₂O, such combination being able to form one or more hydrates.

In another aspect, the present invention also relates to methods of producing an antimicrobial compound of the invention. All embodiments described above for the compound of the invention are also encompassed in this aspect.

Generally, the methods comprise providing the core peptide and attaching a fatty acid moiety to the core peptide, preferably through a bifunctional linker. Synthesis may be biological, chemical, enzymatic, genetic, or a combination thereof. For instance, the peptide may be produced biologically and the fatty acid moiety attached chemically.

In a first embodiment, the method of producing an antimicrobial compound of the invention comprises culturing a microorganism producing said antimicrobial compound under conditions suitable to produce said compound, and optionally recovering said antimicrobial compound from the culture.

The microorganism producing the antimicrobial compound may naturally produce said compound or may be genetically modified to produce said compound.

In an embodiment, the microorganism producing the antimicrobial compound is a microorganism naturally producing said antimicrobial compound.

Preferably, the microorganism is selected from the group consisting of Nocardia, Microbacterium, Tsukamurella, Streptomyces, Nocardiopsis and Nonomuraea bacteria.

More preferably, the microorganism is selected from the group consisting of Nocardia terpenica, preferably Nocardia terpenica DSMZ 44935, Nocardia altamirensis, preferably Nocardia altamirensis DSMZ 44997, Microbacterium arborescens, preferably Microbacterium arborescens CIP 55.81T (Collection Institut Pasteur) or Microbacterium arborescens strain ND21 (NCBI BioSample: SAMN05211039), Microbacterium sp. TS-1 (Uniprot taxon identifier: 1344956), Tsukamurella sp. 1534 (Oulmi et al. J Bacteriol. 2012 October; 194(19): 5482-5483), Streptomyces aureus, preferably Streptomyces aureus DSM 41785, Streptomyces flavochromogenes, preferably Streptomyces flavochromogenes DSM 40541, Streptomyces natalensis, preferably Streptomyces natalensis DSM 40357, Nocardiopsis chromatogenes, preferably Nocardiopsis chromatogenes DSM 44844 and Nonomuraea candida, preferably Nonomuraea candida DSM 45086.

In a particular embodiment, the microorganism is selected from the group consisting of Tsukamurella sp. 1534 (Oulmi et al. J Bacteriol. 2012 October; 194(19): 5482-5483), Streptomyces aureus, preferably Streptomyces aureus DSM 41785, Streptomyces flavochromogenes, preferably Streptomyces flavochromogenes DSM 40541, Streptomyces natalensis, preferably Streptomyces natalensis DSM 40357, Nocardiopsis chromatogenes, preferably Nocardiopsis chromatogenes DSM 44844 and Nonomuraea candida, preferably Nonomuraea candida DSM 45086.

In another embodiment, the microorganism is selected from the group consisting of Nocardia terpenica, preferably Nocardia terpenica DSMZ 44935, Nocardia altamirensis, preferably Nocardia altamirensis DSMZ 44997, Microbacterium arborescens, preferably Microbacterium arborescens CIP 55.81T (Collection Institut Pasteur) or Microbacterium arborescens strain ND21 (NCBI BioSample: SAMN05211039), and Microbacterium sp. TS-1 (Uniprot taxon identifier: 1344956).

In a more particular embodiment, the microorganism is selected from the group consisting of Nocardia terpenica, preferably Nocardia terpenica DSMZ 44935, Nocardia altamirensis, preferably Nocardia altamirensis DSMZ 44997, and Microbacterium arborescens, preferably Microbacterium arborescens CIP 55.81T (Collection Institut Pasteur).

In a particular embodiment, the microorganism is a strain of Nocardia terpenica, preferably Nocardia terpenica DSMZ 44935, and the antimicrobial compound is of formula (I) wherein X₁ is A, X₂ is I, X₃ is S, X₄ is N and X₅ is G, preferably is NOC1 compound (as defined above and in example 1).

In another particular embodiment, the microorganism is a strain of Nocardia altamirensis, preferably Nocardia altamirensis DSMZ 44997, and the antimicrobial compound is of formula (I) wherein X₁ is A, X₂ is I, X₃ is S, X₄ is N and X₅ is G, preferably is NOC2, 3, 4, 5, 6, 7, 8, 9 and/or 10 (as defined above and in example 1).

In a further particular embodiment, the microorganism is a strain of Microbacterium, preferably Microbacterium arborescens, more preferably Microbacterium arborescens CIP 55.81T, and the antimicrobial compound is of formula (I) wherein X₁ is A, X₂ is L, X₃ is G, X₄ is Q and X₅ is S, preferably is compound A, B and/or C (as defined in example 2).

In another particular embodiment, the microorganism is a Tsukamurella strain, preferably Tsukamurella sp. 1534, and the antimicrobial compound(s) is(are) of formula (I) wherein X₁ is A, X₂ is V, X₃ is S, X₄ is S and X₅ is G.

In another particular embodiment, the microorganism is a Streptomyces strain, preferably Streptomyces aureus or Streptomyces flavochromogenes, more preferably Streptomyces aureus DSM 41785 or Streptomyces flavochromogenes DSM 40541, and the antimicrobial compound(s) is(are) of formula (I) wherein X₁ is A, X₂ is G, X₃ is S, X₄ is E and X₅ is G.

In another particular embodiment, the microorganism is a Streptomyces strain, preferably Streptomyces natalensis, more preferably Streptomyces natalensis DSM 40357, and the antimicrobial compound(s) is(are) of formula (I) wherein X₁ is A, X₂ is T, X₃ is S, X₄ is D and X₅ is G.

In another particular embodiment, the microorganism is a Nocardiopsis strain, preferably Nocardiopsis chromatogenes, more preferably Nocardiopsis chromatogenes DSM 44844, and the antimicrobial compound(s) is(are) of formula (I) wherein X₁ is A, X₂ is T, X₃ is A, X₄ is D and X₅ is G.

In another particular embodiment, the microorganism is a Nonomuraea strain, preferably Nonomuraea candida, more preferably Nonomuraea candida DSM 45086, and the antimicrobial compound(s) is(are) of formula (I) wherein X₁ is A, X₂ is A, X₃ is S, X₄ is E and X₅ is T.

In another embodiment, the microorganism producing the antimicrobial compound is a microorganism genetically modified to produce said compound.

RiPP biosynthesis is typically initiated with a ribosomally generated precursor peptide encoded by a structural gene. This precursor peptide usually contains an N-terminal leader peptide fused to a core peptide which is then cyclized. After cyclization, the leader peptide is removed by proteolytic/enzymatic cleavage.

In a particular embodiment, the compound is produced using a microorganism containing a gene encoding the precursor peptide. The gene may be endogenous to the microorganism, or mutated, or a heterologous gene introduced into said microorganism.

The sequence of the precursor of the antimicrobial compound A, B or C produced by Microbacterium arborescens is

(SEQ ID NO: 1)
MSLEQLEALDASSEAAEMAASLGSQSC,

wherein the core peptide is underlined. The sequence of the precursor of the antimicrobial compound NOC1 produced by Nocardia terpenica is

(MIDVTNIAELHELDSTSASAELVASISSNGC; SEQ ID NO: 2),

wherein the core peptide is underlined. The sequence of the precursor of the antimicrobial compounds NOC2 to NOC10 produced by Nocardia altamirensis is

(MIDVTNIADLHDIDATSGAAELVASISSNGC; SEQ ID NO: 3),

wherein the core peptide is underlined. The sequence of the precursor of the antimicrobial compound(s) of formula (I) produced by Tsukamurella sp. 1534 is

(MIDVTDINSLQAIESHSATSELLASVSSSGC; SEQ ID NO: 8),

wherein the core peptide is underlined. The sequence of the precursor of the antimicrobial compound(s) of formula (I) produced by Streptomyces aureus or Streptomyces flavochromogenes is

(MDLTNVIDLQGTEIVADGVELPASGSSEGC; SEQ ID NO: 9),

wherein the core peptide is underlined. The sequence of the precursor of the antimicrobial compound(s) of formula (I) produced by Streptomyces natalensis is

(MDLTNVMELQGTEIVADGVELPASTSSDGC; SEQ ID NO: 10),

wherein the core peptide is underlined. The sequence of the precursor of the antimicrobial compound(s) of formula (I) produced by Nocardiopsis chromatogenes is

(MDIADVMDLQGEEVVADGVELPASTASDGC; SEQ ID NO: 11),

wherein the core peptide is underlined. The sequence of the precursor of the antimicrobial compound(s) of formula (I) produced by Nonomuraea candida is

(MDLANVMDLQGTEIVADGIELPASASSETC; SEQ ID NO: 12),

wherein the core peptide is underlined. The sequence of the precursor of the antimicrobial compound(s) of formula (I) produced by Microbacterium arborescens strain ND21 is

(MTLEQLEALDASSEAAEMAASLGSQSC; SEQ ID NO: 13),

wherein the core peptide is underlined.

In a particular embodiment, the compound is produced using a microorganism containing a gene encoding the precursor peptide selected from any of SEQ ID NOs: 1-3 and 8 to 13. In a more particular embodiment, the compound is produced using a microorganism containing a gene encoding the precursor peptide selected from any of SEQ ID NOs: 1-3 and 13, preferably from any of SEQ ID NOs: 1-3. In another more particular embodiment, the compound is produced using a microorganism containing a gene encoding the precursor peptide selected from any of SEQ ID NOs: 8-13, preferably from any of SEQ ID NOs: 8-12.

In another particular embodiment, the compound is produced using a microorganism containing a gene encoding a precursor peptide selected from variants of any of SEQ ID NOs: 1-3 and 8 to 13. In a more particular embodiment, the compound is produced using a microorganism containing a gene encoding a precursor peptide selected from variants of any of SEQ ID NOs: 1-3 and 13, preferably from variants of any of SEQ ID NOs: 1-3. In another more particular embodiment, the compound is produced using a microorganism containing a gene encoding a precursor peptide selected from variants of any of SEQ ID NOs: 8-13, preferably from any of SEQ ID NOs: 8-12.

Examples of such variants include, but are not limited to

(SEQ ID NO: 4)
MIDVTNIADLHDIDATSGAAELVGSISSNGC
and
(SEQ ID NO: 5)
MIDVTNIADLHDIDATSGAAELVASLSSQGC,
(SEQ ID NO: 6)
MSLEQLEALDASSEAAEMAGSISSNGC,
and
(SEQ ID NO: 7)
MSLEQLEALDASSEAAEMAASLSSQGC.

Based on these sequences and the general knowledge of the skilled person, the gene encoding said precursors may be mutated in order to change the amino acid sequence of the core peptide. The mutation may be easily determined by the skilled person based on codon usage. Preferably, the leader sequence is not mutated. More preferably, the leader sequence corresponds to the leader sequence of the antimicrobial compound precursor naturally produced by the host microorganism.

Alternatively, a heterologous gene encoding the precursor may be introduced and expressed in a host microorganism.

In preferred embodiments, the host microorganism is naturally capable of producing an antimicrobial compound of the invention. Said microorganism may be selected from the group consisting of Nocardia, Microbacterium, Tsukamurella, Streptomyces, Nocardiopsis and Nonomuraea bacteria, preferably from Microbacterium arborescens, Nocardia terpenica, Nocardia altamirensis, Tsukamurella sp. 1534, Streptomyces aureus, Streptomyces flavochromogenes, Streptomyces natalensis, Nocardiopsis chromatogenes and Nonomuraea candida.

In some preferred embodiments, Said microorganism is preferably selected from Microbacterium and Nocardia bacteria, more preferably from Microbacterium arborescens, Nocardia terpenica and Nocardia altamirensis, and even more preferably is Microbacterium arborescens.

In some other embodiments, said microorganism is preferably selected from Tsukamurella, Streptomyces, Nocardiopsis and Nonomuraea bacteria, more preferably from Tsukamurella sp. 1534, Streptomyces aureus, Streptomyces flavochromogenes, Streptomyces natalensis, Nocardiopsis chromatogenes and Nonomuraea candida.

Suitable culture conditions such as medium, temperature and aeration parameters, may be easily defined by the skilled person according to the nature of the cultured microorganism.

Preferably, the antimicrobial compound of the invention is recovered from the culture supernatant. Extraction of said antimicrobial compound from the culture, and in particular from the supernatant, may be performed by any method known by the skilled person, for example by liquid-liquid extraction with an organic solvent such as butanol as illustrated in the experimental section.

The method may further comprise purifying said compound. The compound may be purified by any method known by the skilled person, for example using HPLC as illustrated in the experimental section, ion exchange chromatography, gel electrophoresis, affinity chromatography and the like.

Optionally, the method may also further comprise subjecting the antimicrobial compound to chemical and/or enzymatic modifications.

In particular, the modification may be a deacylation removing/cleaving the lipophilic moiety, i.e. R₁-Y-L, attached to the core peptide, thereby providing a core peptide of formula (VIII)

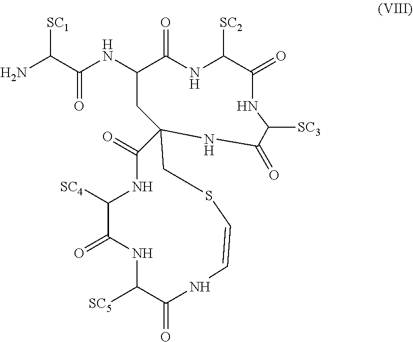

wherein SC₁, SC₂, SC₃, SC₄ and SC₅ represent the side-chains of the amino acids X₁, X₂, X₃, X₄ and X₅, respectively, X₁, X₂, X₃, X₄ and X₅ being as defined above.

This deacylation reaction may be performed by any method known by the skilled person. In particular, this deacylation may be enzymatically performed, preferably using the aculeacin-A deacylase (EC 3.5.1.70) produced by Actinoplanes utahensis NRRL 12052, an enzyme which is routinely used to cleave the fatty acid acyl group of lipopeptides (see e.g. Boeck et al. J Antibiot (Tokyo). 1988 August; 41(8):1085-92). Such deacylation is illustrated in example 3. In this case, the modification step may comprise contacting the antimicrobial compound, preferably the purified compound, with a deacylase or with a microorganism producing said deacylase, preferably with aculeacin-A deacylase of Actinoplanes utahensis NRRL 12052 or with Actinoplanes utahensis NRRL 12052.

Deacylation may be monitored by any analytical method known by the skilled person such as LC-UV or LC-MS analysis.

The core peptide of formula (VIII) may then be isolated or purified by any method known by the skilled person such as HPLC as illustrated in the experimental section, ion exchange chromatography, gel electrophoresis, affinity chromatography and the like.

The core peptide of formula (VIII) may be then reacylated with a R₁—Y-L moiety which is different from the natural one and selected from R₁—Y-L moieties as described above. Acylation may be carried out using any method known by the skilled person. In particular, the core peptide may be contacted with a R₁—Y-L-X moiety, wherein X is halogen, preferably chloride, in the presence of an acylation catalyst, preferably selected from pyridine or 4-dialkylaminopyridines such as 4-dimethylaminopyridine. Such acylation reaction is illustrated in example 3.

Alternatively, or in addition, chemical or enzymatic modifications may also include alteration(s) of the core peptide, in particular of side-chains of amino acids, or of the R₁—Y-L moiety, in particular of the hydrocarbon chain, such as acylation, acetylation or methylation.

The peptide according to the invention may also be obtained by classical chemical synthesis (in solid phase or homogeneous liquid phase) and/or enzymatic synthesis.

In particular, the method of producing an antimicrobial compound of the invention may comprise (i) chemically synthetizing a linear precursor of the core peptide as described above, (ii) contacting said precursor with an enzymatic extract of a microorganism producing an antimicrobial compound of the invention, thereby obtaining a bicyclic core peptide of formula (VIII), and (iii) adding a R₁—Y-L moiety to said bicyclic core peptide, for example by acylation reaction as described above.

The precursor may comprise the leader sequence or not. Preferably, the precursor comprises the leader sequence.

In a further aspect, the present invention also relates to a core peptide of formula (VIII) wherein SC₁, SC₂, SC₃, SC₄ and SC₅ represent the side-chains of the amino acids X₁, X₂, X₃, X₄ and X₅, respectively, X₁, X₂, X₃, X₄ and X₅ being as defined above.

All embodiments described above for the compound of the invention are also encompassed in this aspect.

The present invention also relates to a pharmaceutical composition comprising an antimicrobial compound according to the invention, or any pharmaceutically acceptable salt, solvate or hydrate thereof, and a pharmaceutically acceptable carrier and/or excipient. All embodiments described above for the compound of the invention are also encompassed in this aspect.

The pharmaceutically acceptable excipients and carriers that can be used in the composition according to the invention are well known to one of skill in the art (Remington's Pharmaceutical Sciences, 18^{th} edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3^{rd} edition, A. Kibbe, Ed., Pharmaceutical Press [2000]) and comprise in particular physiological saline solutions and phosphate buffers.

The pharmaceutical composition according to the invention may be suitable for local or systemic administration, in particular for oral, transmucosal (including nasal, rectal or vaginal), topical (including transdermal, buccal and sublingual), or parenteral (including subcutaneous, intramuscular, intravenous and intradermal) administration. For these formulations, conventional excipient can be used according to techniques well known by those skilled in the art.

The compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical composition according to the invention may be formulated to release the active drug substantially immediately upon administration or at any predetermined time or time period after administration.

Pharmaceutical composition according to the invention can comprise one or more antimicrobial compounds of the invention, and one or several pharmaceutically acceptable excipients and/or carriers. These excipients and/or carriers are chosen according to the form of administration as described above.

In an embodiment, the pharmaceutical composition according to the invention comprises from 0.1 mg to 5 g, 1 mg to 2 g, preferably from 10 mg to 1 g, of antimicrobial compound(s) according to the invention.

The pharmaceutical composition according to the invention may also comprise one or several additional active compounds such as other antimicrobial agents. The composition may also additionally comprise substances that can potentiate the activity of the compound according to the invention.

Alternatively, the pharmaceutical composition may consist essentially of one or more antimicrobial compounds of the invention, and one or several pharmaceutically acceptable excipients and/or carriers. As used herein, the term “consist essentially of” indicates that the composition does not comprise other therapeutically active substance in addition to antimicrobial compounds of the invention.

As exemplified in the experimental section, the antimicrobial compounds of the invention can be used in a wide variety of applications to inhibit the growth or kill microorganisms.

In a particular aspect, the present invention relates to an antimicrobial compound of the invention, or any acceptable salt, solvate or hydrate thereof, as a medicament, in particular as a medicament for treating a microbial infection, preferably an infection due to a Gram-positive bacterium. The present invention thus also relates to an antimicrobial compound of the invention, or any acceptable salt, solvate or hydrate thereof, as an antimicrobial agent.

All embodiments described above for the compound of the invention are also encompassed in this aspect.

The medicament may be intended for pharmaceutical or veterinary use.

The term “microbe” or “microbial” as employed herein refers to bacteria, fungi, yeasts, viruses and/or parasites, preferably to bacteria and fungi.

The term “microbial infection” as employed herein refers to an infection caused by bacteria, fungi, yeasts, viruses and/or parasites, preferably caused by bacteria and/or fungi.

The term “antimicrobial activity” as employed herein refers to an antibacterial, antiviral, antifungal and/or antiparasitic activity, preferably an antibacterial and/or antifungal activity. Said activity may be evaluated by measuring different parameters such as IC₅₀ or MIC. Methods to evaluate such activities are well known by the skilled person.

The present invention further relates to an antimicrobial compound of the invention or any pharmaceutically acceptable salt, solvate or hydrate thereof, for use in the treatment or prevention of a microbial infection.

It also concerns the use of a compound of an antimicrobial compound of the invention or any pharmaceutically acceptable salt, solvate or hydrate thereof, for preparing a medicament for treating a microbial infection.

It finally concerns a method for treating a microbial infection in a subject in need thereof, comprising administering an effective amount of an antimicrobial compound of the invention or any pharmaceutically acceptable salt, solvate or hydrate thereof, to the subject.

All embodiments described above for the antimicrobial compound of the invention are also encompassed in this aspect.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

The effective amount may be a therapeutically or prophylactically effective amount. A “therapeutically effective amount” refers to an amount effective, at dosages and for periods of time necessary, to achieve the desired therapeutic or prophylactic result. In particular, this term refers to an amount of antimicrobial compound of the invention administered to a patient that is sufficient to provide an antimicrobial activity on the pathogenic strain responsible for the infection, i.e. that is sufficient to inhibit the growth or to kill the pathogenic strain. The therapeutically effective amount may vary according to various factors such as the physiological condition of the subject to be treated, the severity of the affliction, the administration route, the pathogenic agent and the antimicrobial activity of the compound towards said pathogenic agent. A therapeutically effective amount encompasses an amount in which any toxic or detrimental effects are outweighed by the therapeutically beneficial effects. A “prophylactically effective amount” refers to an amount effective, at dosages and for periods of time necessary, to achieve the desired prophylactic result, i.e. to prevent the microbial infection. Typically, but not necessarily, since a prophylactic dose is used in subjects prior to or at an earlier stage of disease, the prophylactically effective amount would be less than the therapeutically effective amount. Suitable means and measures to determine the therapeutically or prophylactically effective amount are available to the person skilled in the art.

In a particular embodiment, the method of the invention comprises administering from 0.05 to 20 mg/kg of body weight, preferably from 0.1 to 10 mg/kg of body weight, of an antimicrobial compound of the invention, or any acceptable salt, solvate or hydrate thereof, to said subject.

In a more particular embodiment, the method of the invention comprises administering from 0.05 to 20 mg/kg of body weight/day, preferably from 0.1 to 10 mg/kg of body weight/day, of an antimicrobial compound of the invention, or any acceptable salt, solvate or hydrate thereof, to said subject.

The frequency of administration may be for example every 4 to 24 hours, preferably every 8 to 12 hours. The duration of treatment may be for example from 1 to 30 days, preferably from 3 to 20 days, and most preferably from 5 to 10 days.

Preferably, the microbial infection is a bacterial infection, more preferably an infection due to a Gram-positive bacterium, a mycobacterium strain or a fungus.

In an embodiment, the microbial infection is due to a Gram-positive bacterium, preferably selected from the group consisting of bacteria from the Staphylococcus genus such as S. aureus, S. epidermidis, S. saprophiticus, S. lugdunensis, S haemolyticus, S warneri, S schleiferi and S intermedius, the Streptococcus genus such as S. pyogenes, S. agalactiae, S. dysgalactiae, S. bovis, S. anginosus, S. sanguinis, S. suis, S. mitis, S. mutans, S. pneumonia and S. oralis, the Enterococcus genus such as E. faecium, E. faecalis and E. gallinarum, the Listeria genus such as L. monocytogenes and L. ivanovii, the Clostridium genus such as C. perfringens, C. difficile, C. tetani and C. botulinum, the Propionibacterium genus such as P. acnes, P. granulosum, P. avidum and P. propionicus, and Bacillus genus such as Bacillus subtilis.

The Gram-positive bacterium may be a resistant or multi-resistant strain such as methicillin-resistant Staphylococcus aureus (MRSA), methicillin-resistant Staphylococcus epidermidis (MRSE), vancomycin-intermediate Staphylococcus aureus (VISA), vancomycin-resistant Staphylococcus aureus (VRSA), vancomycin-resistant enterococci (VRE) or penicillin-resistant streptococci.

In a particular embodiment, the Gram-positive bacterium is selected from the group consisting of Staphylococcus aureus, epidermidis and saprophiticus (including MRSA, VISA and VRSA, MRSE), Streptococcus pyogenes and pneumonia (including penicillin-resistant S. pyogenes and pneumonia), Listeria ivanovii, Listeria monocytogenes, Enterococcus faecalis and faecium (including vancomycin-resistant E. faecalis and faecium), Clostridium perfringens, Clostridium difficile, Clostridium tetani, Clostridium botulinum and Propionibacterium acnes.

In a more particular embodiment, the Gram-positive bacterium is selected from the group consisting of methicillin sensitive and resistant Staphylococcus aureus and Staphylococcus epidermidis, vancomycin sensitive and resistant Enterococcus faecalis and Enterococcus faecium, Bacillus subtilis, penicillin sensitive and resistant Streptococcus pneumonia, Streptococcus pyogenes, Streptococcus agalactiae, Streptococcus mitis, Streptococcus oralis, Clostridium difficile and Propionibacterium acnes.

In another embodiment, the microbial infection is due to a mycobacterium strain, preferably selected from Mycobacterium leprae and Mycobacterium tuberculosis, preferably Mycobacterium tuberculosis.

In a further embodiment, the microbial infection is due to a pathogenic fungus, preferably selected from the group consisting Candida and Cryptococcus fungi. Examples of Candida fungi include, but are not limited to, Candida albicans, Candida parapsilosis, Candida krusei, Candida glabrata and Candida tropicalis. Examples of Cryptococcus fungi include, but are not limited to, Cryptococcus neoformans.

In the methods of the present invention, the antimicrobial compound of the invention, or any pharmaceutically acceptable salt, solvate or hydrate thereof, may be used in combination with other active ingredients, in particular in combination with other antimicrobial agents. Such combination therapies encompass combined administration (where two or more therapeutic agents are included in the same or separate formulations), and separate administration, in which case, administration of the antimicrobial compound can occur prior to, simultaneously, and/or following, administration of the additional therapeutic agent.

In another aspect, the present invention also relates to the use of the antimicrobial compound according to the invention, or any acceptable salt, solvate or hydrate thereof, as preservative, disinfectant or as phytosanitary agent.

The antimicrobial compound according to the invention may be used as preservative for materials such as foodstuffs, cosmetics, medicaments and other nutrient containing materials. The compound is used in order to eliminate or prevent the risk of infection by microorganisms and thereby improve the conservation of such materials.

The antimicrobial compound according to the invention may also be used as phytosanitary agent, i.e. to prevent or treat infections of plants by phytopathogens. The present invention thus also relates to a method for preventing or treating a plant against phytopathogens comprising contacting said plant with an effective amount of a compound of the invention, or any acceptable salt, solvate or hydrate thereof.

Preferably, phytopathogens are bacterial phytopathogens and more preferably are Gram-positive bacteria such as bacteria belonging to Clavibacter, Streptomyces, Rhodococcus, Bacillus, Clostridium, Arthrobacter, Curtobacterium, Nocardia and Rhodococcus genera.

The antimicrobial compound of the invention may also be used as disinfectant. The term “disinfectant” refers to an antimicrobial activity of the compound on a surface (for example, walls, doors, medical equipment), a liquid (for example, water) or a gas (for example, an anaesthetic gas).

Biofilms are responsible for approximately 60% of nosocomial infections. They are essentially due to microbial colonization of implanted biomaterials. Eradication of a bacterial biofilm is a major clinical problem considering that antibiotics normally active on bacteria in planktonic state often turn out to be much less effective against structures organized into a biofilm.

In an embodiment, the compound according to the invention is used to eliminate bacterial biofilms, preferably biofilms comprising Gram-positive bacteria. In a preferred embodiment, the compound according to the invention is used to disinfect surgical or prosthetic equipment.

The present invention also relates to a food or cosmetic composition comprising at least one compound according to the invention, or any acceptable salt, solvate or hydrate thereof.

It also relates to a phytosanitary composition comprising at least one antimicrobial compound according to the invention, or any acceptable salt, solvate or hydrate thereof, and optionally an acceptable excipient and/or carrier.

These compositions according to the invention can be in solid form such as powder or granules, or in a liquid form.

The present invention also relates to a medical device or implant comprising a body having at least one surface coated with or including an antimicrobial compound according to the invention, or any acceptable salt, solvate or hydrate thereof. The present invention also relates to a method for preparing a medical device or implant comprising applying a coating of antimicrobial compound according to the invention, or any acceptable salt, solvate or hydrate thereof, or placing in contact, with at least one surface of the device or implant.

This type of medical device or implant and the uses and methods of preparation thereof are described for example in patent application WO 2005/006938.

The device or implant may be, for example, intravascular, peritoneal, pleural and urological catheters; heart valves; cardiac pacemakers; vascular shunts; coronary stunts; dental implants or orthopaedic or intraocular prosthesis.

The surface coated with or including an antimicrobial compound of the invention may be composed of thermoplastic or polymeric materials such as polyethylene, Dacron, nylon, polyesters, polytetrafluoroethylene, polyurethane, latex, silicone elastomers and the like, or of metallic materials such as gold. In a particular embodiment, the compound of the invention is covalently attached to a functionalized surface, preferably a metallic surface. Optionally, the antimicrobial compound may be attached to the surface through a spacer arm.

Alternatively, the device or implant, in particular bone and joint prosthetic device, may be coated with a cement mixture comprising an antimicrobial compound according to the invention.

The antimicrobial compound of the invention used on the surface of the medical device or implant may be combined with another active molecule, preferably with another antimicrobial agent.

Further aspects and advantages of the present invention will be described in the following examples, which should be regarded as illustrative and not limiting.

### EXAMPLES

### Example 1

Preparation of Culture Medium for Production of Lipolanthipeptide from Nocardia Bacteria

GYM Medium

The composition of the GYM liquid medium was as follows: 4 g Glucose, 4 g Yeast extract, 10 g Malt extract and 1000 ml distilled water.

The 10% glucose and 3M KOH solutions were prepared separately.

The 10% Glucose (100 ml)

10 g of powder, distilled water qsp 100 mL

Sterilization at 110° C. for 30 minutes

3M KOH (1000 ml)

MM=56.11 g/mol

Purity: 85%

56.11*0.85=47.6 g/mol

Weigh 143.08 g of powder for a qsp of 1 L with distilled water

Autoclave at 121° C. for 20 minutes

GYM Medium Liquid (1000 ml)

4 g yeast extract

10 g malt extract

Sterilization at 121° C. for 20 minutes

40 ml sterile 10% glucose: final concentration 0.4% (final concentration 4 g/L)

Adjust pH to 7.2 using sterile KOH

GYM Medium Agar (1000 ml)

Add CaCO₃ 2.0 g/L and Agar 15.0 g/L

Culture of Nocardia terpernica or Nocardia altamirensis

Preculture (P1)

A 500 ml flask containing as final volume 100 ml GYM medium was inoculated with a colony of the primary Nocardia terpenica (DSMZ 44935) or Nocardia altamirensis (DSMZ 44997) strain and incubated at 30° C. for 24 h with stirring at 240 rotations per minute (rpm). Optical density (OD) at 600 nm was then measured by a spectrophotometer until the Nocardia terpenica or Nocardia altamirensis strain was at the beginning /middle of its exponential growth phase (1<OD at 600 nm<3)

The purity of the pre-culture was monitored by seeding on GYM agar. The plates were incubated at 30° C. for 48 h.

Cultures in Erlenmeyer Flasks

A 5000 ml flask, containing as a final volume 1000 ml GYM medium was inoculated with the 100 ml of pre-culture (P1) and incubated at 30° C. for 96 hours with stirring at 240 rpm. Initial OD at 600 nm ranged between 0.1 and 0.3.

Purity of fermentation was monitored at the end of 96 hours by seeding a GYM agar. The plates were incubated at 30° C. for 48 h.

The culture was centrifuged to 10,000 g for 45 min at 25° C.

The supernatant was recovered and kept at 4° C.

Extraction and Analysis of the Compounds Having Antimicrobial Activity from Nocardia altamirensis

Extraction of the compounds having antimicrobial activity from the supernatant was carried out by liquid-liquid extraction with butanol. Butanol was concentrated to dryness in a rotary evaporator at 50° C. to give the crude extract.

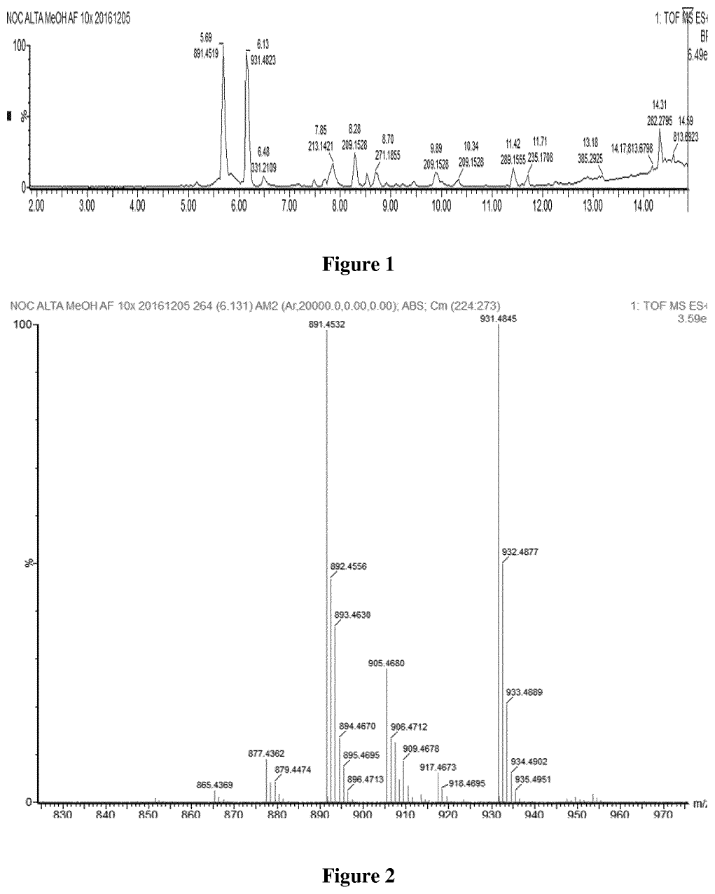

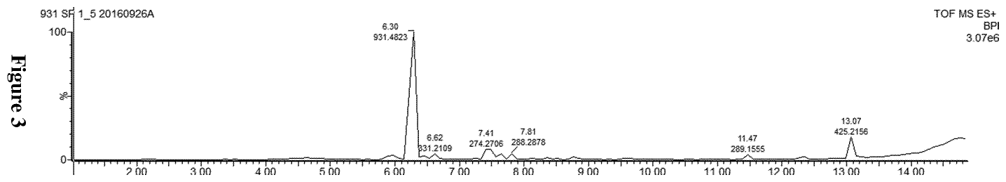

Compounds having antimicrobial activity were analyzed in the crude extract. LC-MS/MS analysis of the crude extract is presented in FIGS. 1 and 2 and Table 1.

LC-MS Conditions

Phenomenex Gemini NX, 5μ, C18, 110 Å, 150×2 mm

Gradient HPLC

Column Gemini C18 5 μm, 100 A 150×2 mm

Solvent A: H20+0.1% formic acid

Solvent B: ACN+0.1% formic acid

Time(min) Flow Rate(mL/min) % A % B

1. Initial 0.500 95.0 5.0

2. 2.00 0.500 95.0 5.0

3. 14.00 0.500 0.0 100.0

4. 15.00 0.500 0.0 100.0

5. 17.00 0.500 95.0 5.0

MS/MS Conditions

MS

Polarity: ES+

Capillary (kV): 3.0000

Source Temperature (° C.): 150

Sampling Cone: 30.0000

Source Offset: 90.0000

Source Gas Flow (mL/min): 0.00

Desolvation Temperature (° C.): 300

Cone Gas Flow (L/Hr): 0.0

Desolvation Gas Flow (L/Hr): 600.0

Nebuliser Gas Flow (Bar): 3.0

Ion Energy: 1.0

Acquisition mass range

Start mass: 100.000

End mass: 2000.000

Function Parameters—Function 1—TOF FAST DDA FUNCTION

[MS SURVEY]

Survey Start Mass: 100.0000

Survey End Mass: 2000.0000

Switch to MS/MS when Intensity rising above threshold

Intensity Threshold: 5000.0

Survey Scan Time: 0.2

Survey Interscan Time: 0.01

Survey Data Format: Continuum

Analyser: Resolution Mode

MS/MS (Automatic Acquisition of 4 Precursor Ions)

MSMS Start Mass: 100.0

MSMS End Mass: 2000.0

Number of precusors: 4

MSMS to MS Switch Criteria: TIC rising above threshold

Switchback threshold: 50000.0

MSMS Switch After Time (sec): 0.2

MSMS Scan Time (sec): 0.10

MSMS Interscan Time (sec): 0.01

MSMS Data Format: Continuum

[COLLISION ENERGY]

Trap MSMS Collision Energy Ramp Low Mass (Da): 100.0

Trap MSMS Collision Energy Ramp High Mass (Da): 2000.0

Trap MSMS Collision Energy Ramp LM Start (eV): 10.0

Trap MSMS Collision Energy Ramp LM End (eV): 20.0

Trap MSMS Collision Energy Ramp HM Start (eV): 70.0

Trap MSMS Collision Energy Ramp HM End (eV): 100.0

[TRANSFER COLLISION ENERGY]

Using MSMS Auto Trap Collision Energy (eV): 2.000000

[CONE VOLTAGE]

Cone (V): 40.0

The antimicrobial compounds isolated from Nocardia altamirensis have the following formula (V)

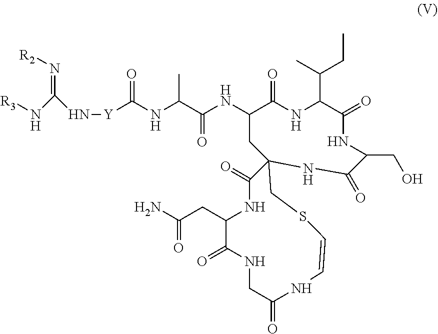

R₂, R₃ and Y being defined in Table 1 below.

TABLE 1
Compounds from Nocardia altamirensis

| | | Molecular | Molecular | |
|---|---|---|---|---|
| Name | Time Retention | weight | formula | HR-(M + H)⁺ |
| NOC2 | 5.64 | 876 | C₃₈H₆₀N₁₂O₁₀S | 877.4365 |
| NOC3 | 5.69 | 890 | C₃₉H₆₂N₁₂O₁₀S | 891.4538 |
| NOC4 | 5.74 | 892 | C₃₉H₆₄N₁₂O₁₀S | 893.4629 |
| NOC5 | 5.74 | 904 | C₄₀H₆₄N₁₂O₁₀S | 905.4678 |
| NOC6 | 5.81 | 906 | C₄₀H₆₆N₁₂O₁₀S | 907.4797 |
| NOC7 | 5.89 | 894 | C₃₉H₆₆N₁₂O₁₀S | 895.4803 |
| NOC8 | 5.95 | 908 | C₄₀H₆₈N₁₂O₁₀S | 909.4958 |
| NOC9 | 6.11 | 916 | C₄₁H₆₄N₁₂O₁₀S | 917.4649 |
| NOC10 | 6.13 | 930 | C₄₂H₆₅N₁₂O₁₀S | 931.4836 |

| | | Y (unsubstituted and uninterrupted | |
|---|---|---|---|
| | | linear hydrocarbon chain) | |
| | | Chain | Number of |
| Name | R₂/R₃ | lenght | double bonds |
| NOC2 | Hydrogen | C10 | 2 |
| NOC3 | hydrogen and methyl | C10 | 2 |
| NOC4 | hydrogen and methyl | C10 | 1 |
| NOC5 | methyl | C10 | 2 |
| NOC6 | methyl | C10 | 1 |
| NOC7 | hydrogen and methyl | C10 | 0 |
| NOC8 | methyl | C10 | 0 |
| NOC9 | hydrogen and methyl | C12 | 3 |
| NOC10 | methyl | C12 | 3 |

Extraction, Purification and Structure of the Antimicrobial Compound Isolated from Nocardia terpenica (NOC1)

Extraction of the compounds having antimicrobial activity from the supernatant was carried out by liquid-liquid extraction with butanol. Crude extract was purified by taking 150 mg in a mixture of H₂O/ACN/DMSO 1/1/1 (v/v/v). The sample was manually loaded (5.0 mL) into the injection system of the semi-preparative HPLC manufactured by Waters. The column used was a C18 (5 microns, 150×10 mm, Gemini, Phenomenex). Elution was performed at a flow rate of 7 mL/min according to the gradient shown in Table 2 below.

TABLE 2
Elution as a function of respective
concentrations of buffers A and B

| Time | Buffer A | Buffer B |
|---|---|---|
| (min) | (H₂O + 0.1% formic acid) | (Acetonitrile + 0.1% formic acid) |
| 0 | 95 | 5 |
| 3 | 90 | 10 |
| 15 | 50 | 50 |
| 16 | 5 | 95 |
| 19 | 5 | 95 |
| 20 | 95 | 5 |

The peak corresponding to compound NOC1 was collected at 10.9 min.

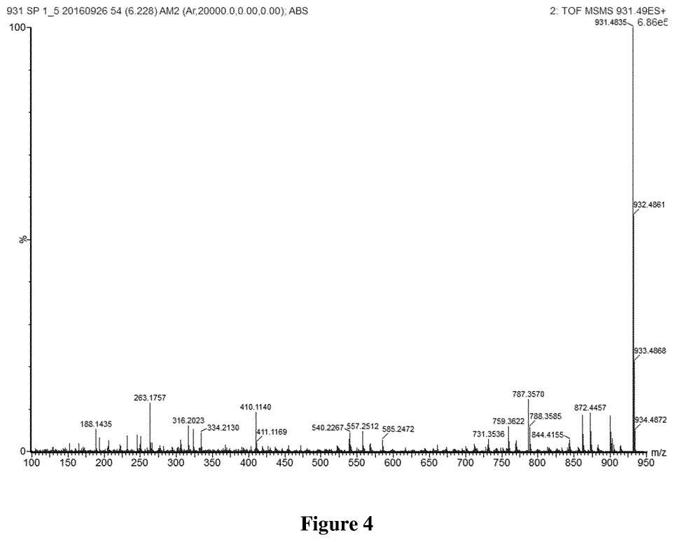

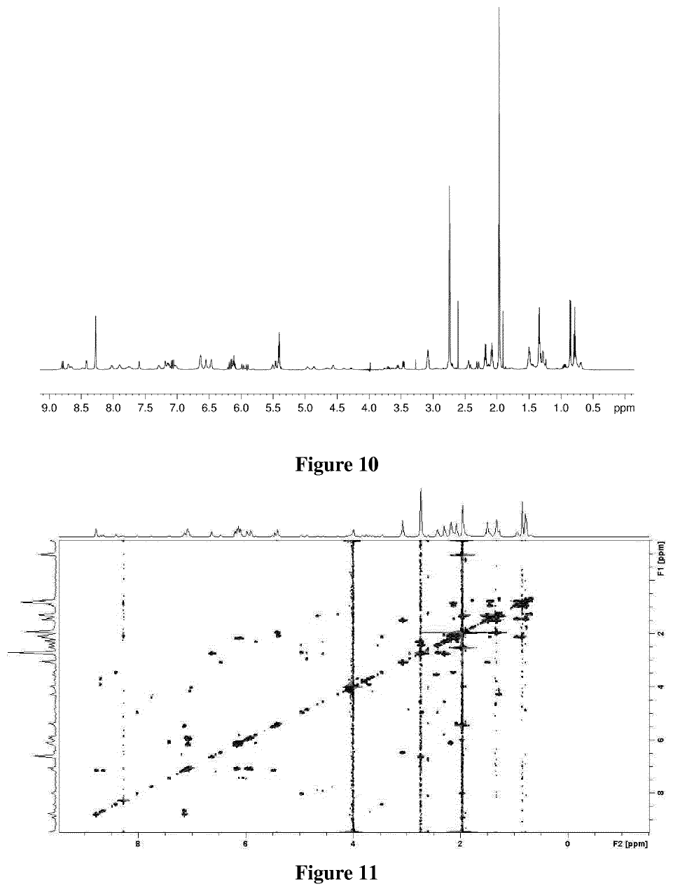

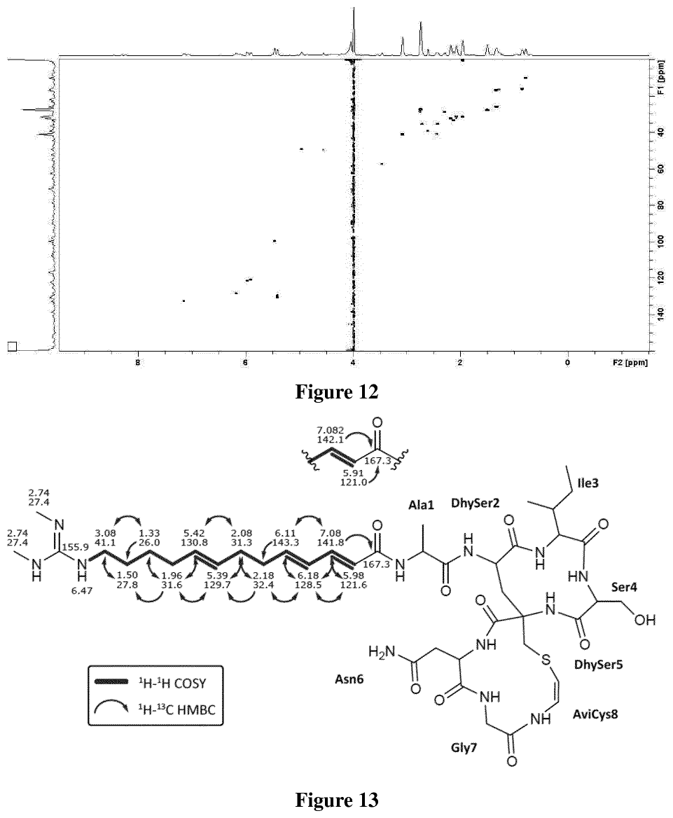

The obtained compound was analyzed by LC-MS/MS analysis (FIG. 3 and FIG. 4) and by NMR 600 Mhz (cf. FIGS. 10 to 13).

According to these analysis, the antimicrobial compound isolated from Nocardia terpenica has the following formula (VI)

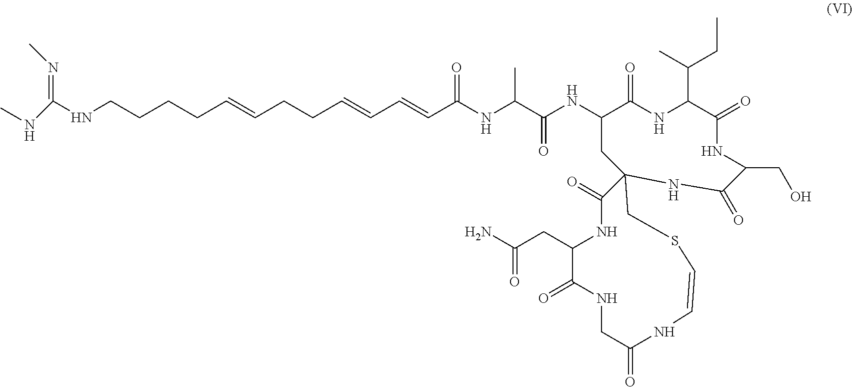 [SMILES: CCC(C)C1NC(=O)C(NC(=O)C(C)NC(=O)/C=C/C=C/CC/C=C/CCCCN/C(=N/C)NC)CC2(CS/C=C\NC(=O)CNC(=O)C(CC(N)=O)NC2=O)NC(=O)C(CO)NC1=O]

Antibacterial Activities of the Compound NOC1

The measures of activities were conducted on compound NOC1 from Nocardia terpenica following the protocol recommended by the Clinical and Laboratory Standards Institute (CLSI)—Clinical and Laboratory Standards Institute (CLSI, formerly NCCLS): Methods for Dilution Antibacterial Susceptibility Tests for Bacteria That Grow Aerobically; Approved Standard—Tenth Edition (2015). Clinical and Laboratory Standards Institute Document M07-A10.

The activities are illustrated in tables 3 and 4 hereafter.

TABLE 3
MIC for compound NOC1 against different
strains of Staphylococcus aureus

| | Minimal Inhibitory |
|---|---|
| | Concentration |
| Strain | (MIC) μg/mL |
| S. aureus - ATTC 13709 (Fully susceptible) | 2 |
| S. aureus - ATTC 1683 (Methicillin resistant) | 16 |
| S. aureus - ATTC 25923 | 2 |
| S. aureus - USA300 | 60 |

TABLE 4
Extended antimicrobial activities of compound NOC1

| | | Characterized | MIC |
|---|---|---|---|
| Strain ID | Strain | Resistance | (μg/mL) |
| Gram-positive | | | |
| Aerobe: | | | |
| ATCC13709 | Staphylococcus aureus | Methicillin | 2 |
| | | sensitive | |
| ATCC1683 | Staphylococcus aureus | Methicillin | 16 |
| | | resistant | |
| ATCC25923 | Staphylococcus aureus | Methicillin | 2 |
| | | sensitive | |
| USA300 | Staphylococcus aureus | Methicillin | 60 |
| | | resistant | |
| ATCC29212 | Enterococcus faecalis | Vancomycin | 160 |
| | | sensitive | |
| ATCC700802 | Enterococcus faecalis | Vancomycin | 16 |
| | | resistant | |
| | | (gene vanB) | |
| ATCC19434 | Enterococcus faecium | Vancomycin | 8 |
| | | sensitive | |
| ATCC51858 | Enterococcus faecium | Vancomycin | 16 |
| | | resistant | |
| | | (gene vanB) | |
| ATCC51559 | Enterococcus faecium | Vancomycin | 16 |
| | | resistant | |
| | | (gene vanA) | |
| ATCC6633 | Bacillus subtilis | | 2 |
| Fungi: | | | |
| ATCC10231 | Candida albicans | | 3 |
| DSMZ5784 | Candida parapsilosis | | 24 |
| DSMZ6128 | Candida krusei | | 12 |
| DSMZ6425 | Candida glabrata | | 12 |
| DSMZ6972 | Cryptococcus neoformans | | 24 |
| DSMZ11953 | Candida tropicalis | | 6 |

### Example 2

Preparation of Culture Medium for Production of Lipolanthipeptide from Microbacterium bacteria

YPG (Peptone, Glucose, Yeast Extract) Medium

The composition of the YPG medium is as follows: glucose, 1 g/L; peptone, 10 g/L; yeast extract, 5 g/L; MOPS (3-(N-morpholino)propansulfonic acid) 150 mM

The 10% glucose, 2M MOPS and 3M KOH solutions are prepared separately.

YPG Medium
10 g/L of peptone
5 g/L yeast extract

Sterilization at 121° C. for 20 minutes

Addition of sterile 10% glucose: final concentration 0.1% (final concentration 1 g/L)

Addition of sterile MOPS (final concentration 150 mM)

Adjust pH to 7.2 using sterile KOH or sterile KCl depending on the initial pH.

Culture of Microbacterium arborescens CIP 55.81T.

Pre-Culture (P1)

A 500 ml flask containing as final volume 100 ml YPG medium was inoculated with a colony of the primary Microbacterium arborescens strain bank and incubated at 30° C. for 24 h with stiffing at 160 rotations per minute (rpm). Optical density (OD) at 600 nm was then measured by a spectrophotometer until the Microbacterium arborescens strain was at the beginning/middle of its exponential growth phase (1 <OD at 600 nm <3).

The purity of the pre-culture was monitored by seeding on YPG agar. The plates were incubated at 30° C. for 48 h.

Cultures in Erlenmeyer Flasks

A 5000 ml flask, containing as a final volume 1000 ml YPG medium was inoculated with the 100 ml of pre-culture (P1) and incubated at 30° C. for 96 hours with stirring at 160 rpm. Initial OD at 600 nm ranged between 0.1 and 0.3.

Purity of fermentation was monitored at the end of 96 hours by seeding a YPG agar. The plates were incubated at 30° C. for 48 h.

The culture was centrifuged to 10,000 g for 45 min at 25° C.

The supernatant was recovered and kept at 4° C.

Extraction of Lipolanthipeptide

Extraction of the compounds having antimicrobial activity from the supernatant was carried out by liquid-liquid extraction in contact with a mixture of dichloromethane/methanol in a 80:20 ratio. The operation is carried out 5 times using the collected supernatant. The solvent was concentrated to a final volume of 20 ml in a rotary evaporator at 50° C., 7 mbar, 160 rpm. A precipate was formed, the supernatant was taken off and the precipitate (brown) (PRE1) was redissolved in methanol and the solvent was evaporated under vacuum.

PRE1 was washed several times with dichloromethane then with dichloromethane/Methanol (99/1) to obtain precipitate 2 (yellow) (PRE2).

Purification by Preparative HPLC

PRE2 was purified by taking 150 mg in a mixture of DMSO, H2O, acetonitrile 1/1/1 (v/v/v). The sample was manually loaded (1.5 mL) into the injection system of the semi-preparative HPLC manufactured by Waters. The column used was a C18 (5 microns, 150×21 mm, Gemini, Phenomenex). Elution was performed at a flow rate of 15 mL/min according to the gradient shown in Table 1 below:

TABLE 5
Elution as a function of respective
concentrations of buffers A and B

| Time | Buffer A | Buffer B |
|---|---|---|
| (min) | (H₂O) | (Acetonitrile + 0.1% formic acid) |
| 0 | 100 | 0 |
| 2 | 100 | 0 |
| 17 | 50 | 50 |
| 19 | 0 | 100 |
| 23 | 0 | 100 |
| 25 | 100 | 0 |
| 30 | 100 | 0 |

The three peaks corresponding to compounds A, B and C were collected at 15.1 min, 15.8 min and 16.3 min respectively.

The obtained compounds were analyzed by MALDI-TOF mass spectrometry and by NMR.

The general structure of compounds A, B and C was as follow:

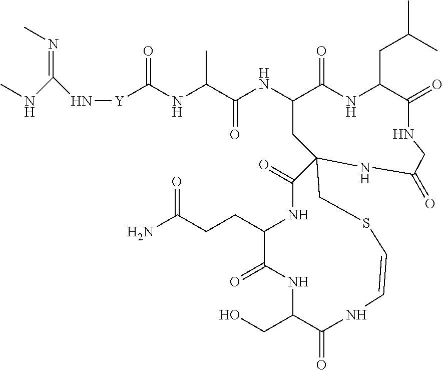

wherein

for compound A: Y is —(CH₂)₁₄—,

for compound B: Y is —(CH₂)₄—(CH₂)ₘ—CH═CH—(CH₂)ₙ—, wherein m and n are independently selected from 0 and integers from 1 to 10, and m+n=10; and

for compound C: Y is —(CH₂)₁₆—.

Antimicrobial Activities of Compounds A, B and C

The measures of activities were conducted on compounds A, B and C, following the protocol recommended by the Clinical and Laboratory Standards Institute (CLSI)—Clinical and Laboratory Standards Institute (CLSI, formerly NCCLS):

Methods for Dilution Antibacterial Susceptibility Tests for Bacteria That Grow Aerobically; Approved Standard—Tenth Edition (2015). Clinical and Laboratory Standards Institute Document M07-A10.

Methods for Antimicrobial Susceptibility Testing of Anaerobic Bacteria; Approved Standard—Eighth Edition (2012). Clinical and Laboratory Standards Institute Document M11-A8.

Antimycobacterial activity was determined as described in Journal of Clinical Microbiology (2009, 47:1773-1780) by Springer et al. Quantitative drug susceptibility testing of Mycobacterium tuberculosis by use of MGIT 960 and EpiCenter Instrumentation.

The activities are illustrated in tables 6 and 7 hereafter.

TABLE 6
MIC for compounds A, B and C against different
strains of Staphylococcus aureus

| | Minimal Inhibitory Concentration | | |
|---|---|---|---|
| | (MIC) μg/mL | | |
| Strain | A | B | C |
| S. aureus - ATTC 13709 | ≤0.04 | ≤0.04 | ≤0.04 |
| (Fully susceptible) | | | |
| S. aureus - ATTC 1683 | ≤0.3 | ≤0.3 | ≤0.3 |
| (Methicillin resistant) | | | |
| S. pneumoniae - ATTC 33400 | ≤0.15 | ≤0.15 | ≤0.08 |
| S. aureus - USA300 | ≤0.08 | ≤0.08 | ≤0.08 |

TABLE 7
Extended antimicrobial activities of compound C

| | | | |
|---|---|---|---|
| | | Characterized | MIC |
| Strain ID | Strain | Resistance | (μg/mL) |

Gram-positive Aerobe:

| | | | |
|---|---|---|---|
| ATTC13709 | Staphylococcus aureus | Methicillin | ≤0.04 |
| | | sensitive | |
| ATCC1683 | Staphylococcus aureus | Methicillin | ≤0.3 |
| | | resistant | |
| 37361192 | Staphylococcus epidermidis | Methicillin | ≤0.25 |
| | | sensitive | |
| 31435861 | Staphylococcus epidermidis | Methicillin | ≤0.25 |
| | | resistant | |
| 31432663 | Streptococcus agalactiae | | ≤0.25 |
| 37352281 | Streptococcus pyogenes | | ≤0.5 |
| 39050149 | Streptococcus mitis | | ≤0.25 |
| 39151368 | Streptococcus oralis | | ≤0.25 |
| R119 | Streptococcus pneumoniae | Penicillin | ≤0.125 |
| (R6 like) | | sensitive | |
| 6883 | Streptococcus pneumoniae | Penicillin | ≤0.125 |
| | | resistant | |
| ATTC1858 | Enterococcus faecium | Vancomycin | ≤0.5 |
| | | sensitive | |
| 31152980 | Enterococcus faecium | Van A | ≤0.25 |
| | | resistant | |
| 31430797 | Enterococcus faecium | Van B | ≤0.25 |
| | | resistant | |

Gram-positive Anaerobe:

| | | |
|---|---|---|
| ATTC700057 | Clostridium difficile | ≤0.25 |
| 1201 | Propionibacterium acnes | ≤0.06 |

Mycobacteria :

| | | |
|---|---|---|
| H37Rv | M. tuberculosis | ≤1 |

### Example 3

The lipophilic moiety of compound A described in example 2 was eliminated by enzymatic deacylation and replaced by stearoyl chain.

Preparation of the Bicyclic Core Peptide

A. Fermentation of Actinoplanes utahensis

A stock culture of Actinoplanes utahensis NRRL 12052 is prepared and maintained on an agar slant. The medium used to prepare the slant is selected from one of the following:

TSB MEDIUM: Tryptic Soy Broth Formula Per Liter Purified Water: Tryptone (Pancreatic Digest of Casein) 17.0 g, Soytone (Peptic Digest of Soybean Meal) 3.0 g, Glucose (=Dextrose) 2.5 g, Sodium Chloride 5.0 g, Dipotassium Hydrogen Phosphate 2.5 g, pH 7.3±0.2.

The pH of the medium was about 7.0 after sterilization by autoclaving at 121° C. for 20 minutes.

The slant was inoculated with Actinoplanes utahensis NRRL 12052, and the inoculated slant was incubated at 30° C. for about 8 to 10 days. The slant was used to inoculate 100 ml of TSB MEDIUM.

TSB MEDIUM was incubated in a 500-ml baffle Erlenmeyer flask at 30° C. for about 72 hours on a shaker at 240 RPM.

In order to provide a larger volume of inoculum, 100 ml of the incubated TSB medium 100 ml was used to inoculate 900 ml of TSB medium having the same composition as the TSB medium 100 ml.

TSB MEDIUM 1000 ml was incubated in a 5000 ml baffle Erlenmeyer flask at 30° C. for about 120 hours on a shaker at 240 RPM.

B. Concentration of the Enzyme

Whole fermentation broth was centrifugated for 45 min at 10000 g. The supernatant (100 ml) was concentrated with Centricon Plus-70 Centrifugal Filter, Ultracel-PL Membrane, 30 kDa from Millipore. The filtrate (5 ml) thus obtained was collected.

C. Deacylation of Compound A

Reaction mixture:

0.85 mg of purified compound A was added to 0.65 ml of distilled water to obtain a concentration of 1.3 mg/ml

0.35 ml of PBS 0.1 M

0.25 ml of concentrated enzyme as described in section B

Temperature of 45° C. under magnetic agitation

Deacylation of compound A was monitored by HPLC analysis with a UV monitor at 254 nm.

The column used was a C18 (5 microns, 150×2 mm, Gemini, Phenomenex). Elution was performed at a flow rate of 0.5 mL/min according to the gradient shown in Table 8 below:

TABLE 8
Elution as a function of respective
concentrations of buffers A and B

| Time | Buffer A | Buffer B |
|---|---|---|
| (min) | (H2O + 0.1% formic acid) | (Acetonitrile + 0.1% formic acid) |
| 0 | 95 | 5 |
| 8 | 20 | 80 |
| 9 | 0 | 100 |
| 10 | 0 | 100 |
| 11 | 95 | 5 |

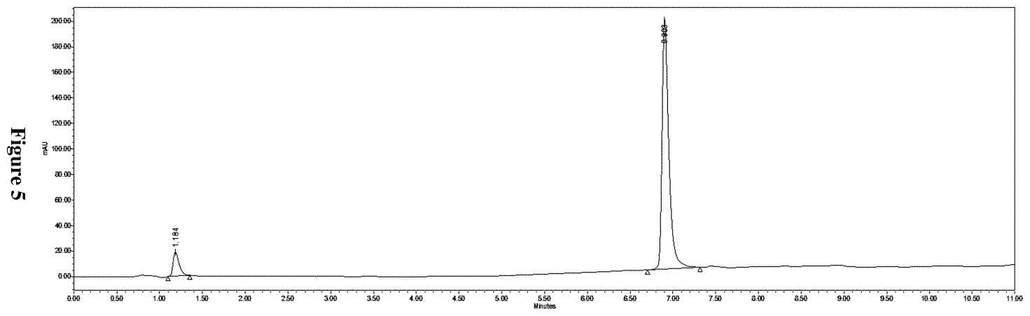

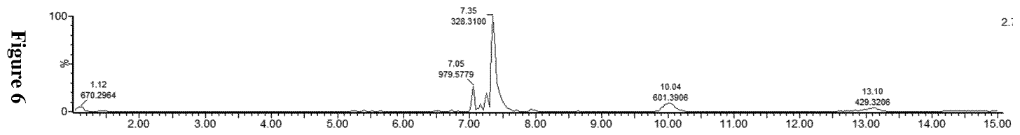

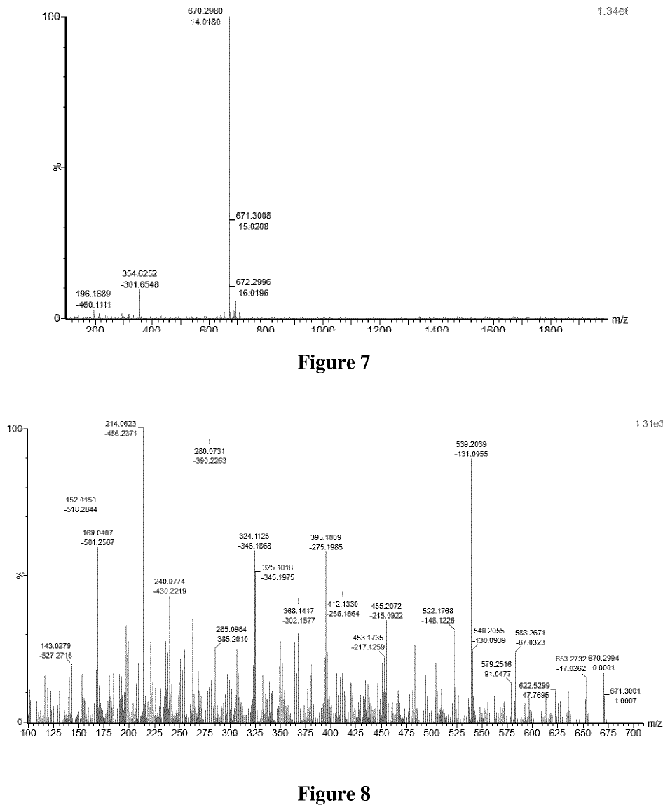

LC-UV and LC-MS/MS analysis of the reaction mixture is presented in FIGS. 5 to 8.

D. Isolation of the Core Peptide

Reaction mixture was briefly centrifugated (8000 g-5 min) and the core peptide was purified by taking 1 ml of the reaction mixture. The sample was manually loaded into the injection system of the semi-preparative HPLC manufactured by Waters. The column used was a C18 (5 microns, 150×10 mm, Gemini, Phenomenex). Elution was performed at a flow rate of 7 mL/min according to the gradient shown in Table 9 below:

TABLE 9
Elution as a function of respective
concentrations of buffers A and B

| Time | Buffer A | Buffer B |
|---|---|---|
| (min) | (H₂O + 0.1% formic acid) | (Acetonitrile + 0.1% formic acid) |
| 0 | 98 | 2 |
| 1 | 98 | 2 |
| 8 | 20 | 80 |
| 9 | 10 | 90 |
| 10 | 10 | 90 |
| 11 | 98 | 2 |

The peak corresponding to the core peptide was collected at 5.8 min, concentrated under vacuum to remove the acetonitrile to give about 100 μg of purified core peptide.

E. Acylation of the Core Peptide with Stearoyl Chloride

To a solution of 600 μg of purified core peptide prepared as above in DMF (450 μL) was added successively pyridine (450 μL), a few amount of 4-dimethylaminopyridine and stearoyl chloride (300 μL). The reaction mixture was stirred at room temperature for 2 h and then concentrated to dryness to give a mixture of the formyl core peptide (RT=4.32 min, MH+=698,2936) and the desired compound (RT=25.26 min, MH+=936,5596) represented below.

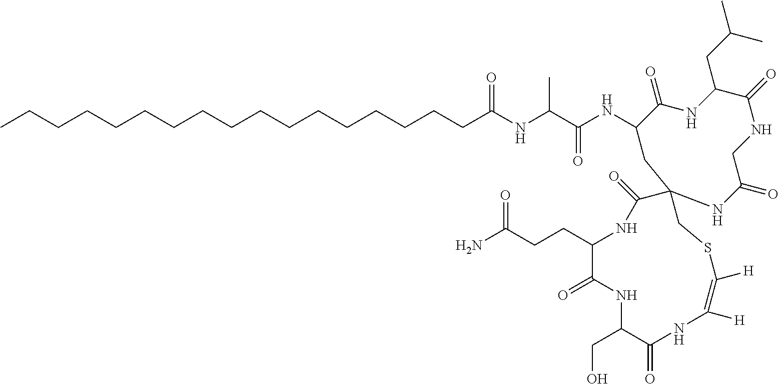 [SMILES: CCCCCCCCCCCCCCCCCC(=O)NC(C)C(=O)NC1CC2(CS/C=C\NC(=O)C(CO)NC(=O)C(CCC(N)=O)NC2=O)NC(=O)CNC(=O)C(CC(C)C)NC1=O]

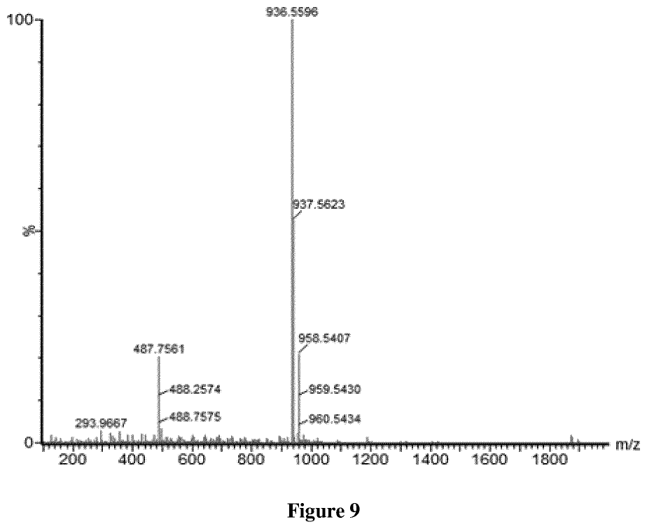

MS spectra of the desired compound is presented on FIG. 9

The molecular formula of said compound is C₄₅H₇₈N₉O₁₀S, and its theoretical mass is 936,55929.

### Example 4

Using bioinformatic tools, the inventors identified several additional microorganisms producing antimicrobial compounds of the invention.

These microorganisms are listed in Table 10 below. For each of these microorganisms, the sequence of the antimicrobial compound precursor is specified.

TABLE 10
List of microorganisms producing an antimicrobial compound of the
invention

| | | Sequence of the antimicrobial |
|---|---|---|
| | Accession number, identifier or | compound precursor |
| Strain | reference | (the core peptide is underlined) |
| Microbacterium arborescens | NCBI BioSample: | MTLEQLEALDASSEAAEMAAS |
| ND21 | SAMN05211039 | LGSQSC (SEQ ID NO: 13) |
| Microbacterium sp. TS-1 | Uniprot taxon identifier: | MSLEQLEALDASSEAAEMAAS |
| | 1344956 | LGSQSC (SEQ ID NO: 1) |
| Tsukamurella sp. 1534 | Oulmi et al. J Bacteriol. | MIDVTDINSLQAIESHSATSELL |
| | 2012 October; | ASVSSSGC (SEQ ID NO: 8) |
| | 194(19): 5482-5483 | |
| Streptomyces aureus | DSM 41785 | MDLTNVIDLQGTEIVADGVELP |
| | | ASGSSEGC (SEQ ID NO: 9) |
| Streptomyces | DSM 40541 | MDLTNVIDLQGTEIVADGVELP |
| flavochromogenes | | ASGSSEGC (SEQ ID NO: 9) |
| Nocardiopsis chromatogenes | DSM 44844 | MDIADVMDLQGEEVVADGVE |
| | | LPASTASDGC (SEQ ID NO: 11) |
| Nonomuraea candida | DSM 45086 | MDLANVMDLQGTEIVADGIEL |
| | | PASASSETC (SEQ ID NO: 12) |
| Streptomyces natalensis | DSM 40357 | MDLTNVMELQGTEIVADGVEL |
| | | PASTSSDGC (SEQ ID NO: 10) |

1. A compound of formula (I) 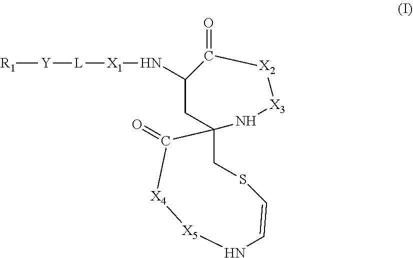
wherein
X₁, X₂, X₃, X₄ and X₅ are independently selected and each represents an amino acid,
L is a bifunctional linker, preferably selected from the group consisting of —C(═O)—, —SO₂—, —C(═S)—, —O—C(═S)—, —NHC(═S)—, —PO—, —OPO—, —OC(═O)— and —NHC(═O)—,
Y is a C₆-C₂₀ saturated or unsaturated linear hydrocarbon chain, said chain being optionally (i) interrupted by one or several heteroatoms independently selected from N, S and O, and/or (ii) interrupted by one or several groups independently selected from a phenyl group and a 5 or 6-membered-ring heterocycle, said phenyl group or heterocycle being optionally substituted by one or several groups independently selected from C₁-C₃ alkyl groups, —OH and C₁-C₃ alkoxy groups, and/or (iii) substituted by one or several groups independently selected from C₁-C₃ alkyl groups, halogens, —OH, methoxy or acetoxy, and
R₁ is selected from the group consisting of hydrogen and a basic group,
or any pharmaceutically acceptable salt, solvate or hydrate thereof.

2. The compound of claim 1, wherein R₁ is selected from the group consisting of hydrogen and a basic group selected from the group consisting of —NR₂R₃, 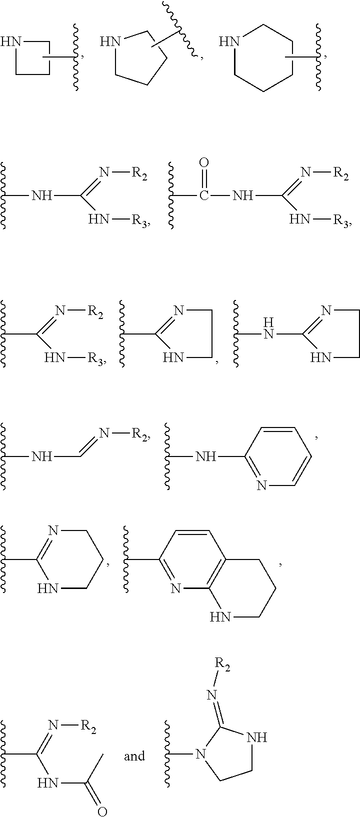
with R₂ and R₃ being independently selected from hydrogen, C₁-C₃ alkyl groups and —C(═O)R₄, and R₄ being a C₁-C₃ alkyl group.

3. The compound of claim 1, wherein R₁ is 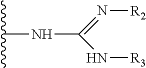
with R₂ and R₃ being independently selected from hydrogen and C₁-C₃ alkyl groups, preferably being methyl.

4. The compound of any of claims 1 to 3, wherein L is —C(═O)—.

5. The compound of any of claims 1 to 4, wherein
a) X₁ is an amino acid selected from the group consisting of A, G, Q, L, W, S and T, preferably A or G, more preferably A; and/or
b) X₂ is an amino acid selected from the group consisting of R, L, V, I, G, T, A, and S, preferably from L, V, I, G and A, even more preferably from L, V, I and A, more preferably L or I, and even more preferably I; and/or
c) X₃ is an amino acid selected from the group consisting of G, S, A, C, L, V, T, P and I, preferably from G, S, A and T, more preferably G or S, and more preferably S; and/or
d) X₄ is an amino acid selected from the group consisting of I, Q, S, N, E, D, W, H, P and T, preferably Q or N, more preferably N; and/or
e) X₅ is an amino acid selected from the group consisting of G, A, S, T, N, R, H, P and D, preferably from G, A, S and T, more preferably G or S, even more preferably G.

6. The compound of any of claims 1 to 5, wherein
X₁ is an amino acid selected from the group consisting of A and G, preferably is A, and/or
X₂ is an amino acid selected from the group consisting of L, V, I, G, A, R, T and S, preferably from the group consisting of L, V, I, G, A and T, more preferably from the group consisting of L, V, I, G and A, and even more preferably from the group consisting of L or I and/or
X₃ is an amino acid selected from the group consisting of G, A, S and T, preferably from the group consisting of G, A and S, and more preferably from the group consisting of G and S, and/or
X₄ is an amino acid selected from the group consisting of Q, N, I, S, E, D, W, H, P and T, preferably from the group consisting of Q, N, S, E and D, more preferably from the group consisting of Q and N, and/or
X₅ is an amino acid selected from the group consisting of G, A, S and T, preferably from the group consisting of G, S and T, more preferably from the group consisting of G and S.

7. The compound of any of claims 1 to 6, wherein
X₁ is an amino acid selected from the group consisting of A and G, preferably is A, and/or
X₂ is an amino acid selected from the group consisting of L, V, I, G and A, preferably from the group consisting of L or I and/or
X₃ is an amino acid selected from the group consisting of G, A, S and T, preferably from the group consisting of G and S, and/or
X₄ is an amino acid selected from the group consisting of Q and N, and/or
X₅ is an amino acid selected from the group consisting of G, A, S and T, preferably from the group consisting of G and S.

8. The compound of any of claims 1 to 6, wherein
X₁ is an amino acid selected from the group consisting of A and G, preferably is A,
X₂ is an amino acid selected from the group consisting of V, I, G, T and A,
X₃ is an amino acid selected from the group consisting of A and S,
X₄ is an amino acid selected from the group consisting of N, S, E and D, and
X₅ is an amino acid selected from the group consisting of G and T.

9. The compound of any of claims 1 to 7, wherein X₁ is A, X₂ is L, X₃ is G, X₄ is Q and X₅ is S.

10. The compound of any of claims 1 to 8, wherein X₁ is A, X₂ is I, X₃ is S, X₄ is N and X₅ is G.

11. The compound of any of claims 1 to 8, wherein X₁ is A, X₂ is V, X₃ is S, X₄ is S and X₅ is G.

12. The compound of any of claims 1 to 8, wherein X₁ is A, X₂ is T, X₃ is A, X₄ is D and X₅ is G.

13. The compound of any of claims 1 to 8, wherein X₁ is A, X₂ is T, X₃ is S, X₄ is D and X₅ is G.

14. The compound of any of claims 1 to 8, wherein X₁ is A, X₂ is A, X₃ is S, X₄ is E and X₅ is T.

15. The compound of any of claims 1 to 8, wherein X₁ is A, X₂ is G, X₃ is S, X₄ is E and X₅ is G.

16. The compound of any of claims 1 to 9, wherein when X₁ is A, X₂ is L, X₃ is G, X₄ is Q, X₅ is S, L is —C(═O)—, and R₁ is 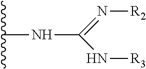
with R₂ and R₃ being methyl,
then Y is not selected from the group consisting of —(CH₂)ₘ— with m being 14 or 16, and a C₁₆ linear hydrocarbon chain comprising one unsaturation, said unsaturation being a double bond.

17. The compound of any of claims 1 to 16, wherein Y is a C₆-C₂₀ saturated or unsaturated linear hydrocarbon chain optionally interrupted by a phenyl group.

18. The compound of any of claims 1 to 17, wherein Y is a C₆-C₁₃ saturated or unsaturated linear hydrocarbon chain, preferably a C₉-C₁₃ saturated or unsaturated linear hydrocarbon chain.

19. The compound of formula (V) 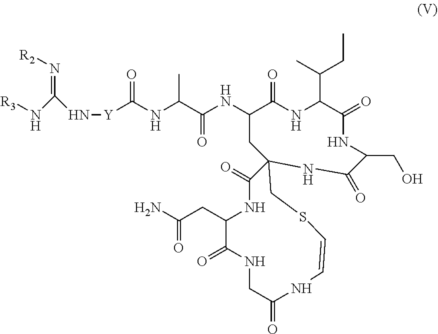
wherein
R₂ and R₃ are hydrogen or methyl and
Y is a C₆-C₁₃ saturated or unsaturated linear hydrocarbon chain, preferably selected from the group consisting of
(i) a C₁₀ saturated linear hydrocarbon chain,
(ii) C₁₀ unsaturated linear hydrocarbon chains comprising one or two double bonds, preferably selected from —(CH₂)₄—(CH₂)ₘ—CH═CH—(CH₂)ₙ—CH═CH—(CH₂)ₚ—, wherein m, n and p are independently selected from 0 and integers from 1 to 2, and m+n+p=2, and —(CH₂)₄—(CH₂)ₘ—CH═CH—(CH₂)ₙ—, wherein m and n are independently selected from 0 and integers from 1 to 4, and m+n=4; and
(iii) C₁₂ unsaturated linear hydrocarbon chains comprising three double bonds, preferably —(CH₂)₄—(CH₂)ₘ—CH═CH—(CH₂)ₙ—CH═CH—(CH₂)ₚ—CH═CH—(CH₂)_{q}—, wherein m, n, p and q are independently selected from 0 and integers from 1 to 2, and m+n+p+q=2, and more preferably —(CH₂)₄—CH═CH—(CH₂)₂—CH═CH—CH═CH.

20. A compound of any of claims 1 to 19, or any acceptable salt, solvate or hydrate thereof, as a medicament.

21. A pharmaceutical composition comprising a compound of any of claims 1 to 19, or any acceptable salt, solvate or hydrate thereof, and a pharmaceutically acceptable carrier and/or excipient.

22. A compound of any of claims 1 to 19, or any acceptable salt, solvate or hydrate thereof, for use in the treatment of a microbial infection, preferably a bacterial or fungal infection.

23. The compound for use of claim 22, wherein the microbial infection is a bacterial infection, preferably an infection due to a Gram-positive bacterium.

24. The compound for use of claim 23, wherein the Gram-positive bacterium is selected from the group consisting of methicillin sensitive and resistant Staphylococcus aureus and Staphylococcus epidermidis, vancomycin sensitive and resistant Enterococcus faecalis and Enterococcus faecium, Bacillus subtilis, penicillin sensitive and resistant Streptococcus pneumonia, Streptococcus pyogenes, Streptococcus agalactiae, Streptococcus mitis, Streptococcus oralis, Clostridium difficile and Propionibacterium acnes.

25. The compound for use of claim 22, wherein the microbial infection is due to a mycobacterium strain, preferably Mycobacterium tuberculosis.

26. The compound for use of claim 22, wherein the microbial infection is due to a pathogenic fungus, preferably selected from the group consisting Candida albicans, Candida parapsilosis, Candida krusei, Candida glabrata and Candida tropicalis and Cryptococcus neoformans.

27. A phytosanitary composition comprising a compound of any of claims 1 to 19 or any acceptable salt, solvate or hydrate thereof, and optionally an acceptable carrier and/or excipient.

28. A method for preventing or treating a plant against phytopathogens, preferably bacteria or fungi, comprising contacting said plant with an effective amount of a compound of any of claims 1 to 19 or any acceptable salt, solvate or hydrate thereof.
